(19) United States
(12) Patent Application Publication
(10) Pub. No.: US 2014/0030744 A1
(43) Pub. Date: Jan. 30, 2014

(54) Means and Methods for Diagnosing Gastric Bypass and Conditions Related Thereto

(71) Applicants: INSERM Institute National de la Sante et de la Recherche Medicale, Paris Cedex 13 (FR); Metanomics GmbH, Berlin (DE)

(72) Inventors: Jens Fuhrmann, Berlin (DE); Dietrich Rein, Berlin (DE); Beate Kamlage, Berlin (DE); Jan C. Wiemer, Berlin (DE); Michael Manfred Herold, Berlin (DE); Karine Clément, Paris (FR); David M. Mutch, Oakville (CA)

(73) Assignees: INSERM Institute National de la Sante et de la Recherche Medicale, Paris Cedex 13 (FR); Metanomics GmbH, Berlin (DE)

(21) Appl. No.: 14/041,432

(22) Filed: Sep. 30, 2013

Related U.S. Application Data
(62) Division of application No. 13/003,314, filed on Jan. 10, 2011.

(30) Foreign Application Priority Data
Jul. 15, 2008 (EP) 08160396.1

(51) Int. Cl. G01N 33/68

(52) U.S. Cl. CPC G01N 33/6893
USPC 435/7.92; 436/501

(57) ABSTRACT

The present invention relates to the field of diagnostic measures. Specifically, it contemplates a method for assessing whether gastric bypass therapy was successful in a subject, a method of predicting whether gastric bypass therapy will be beneficial for a subject in need thereof, and a method of diagnosing whether a supportive therapy accompanying gastric bypass has beneficial effects on a subject in need thereof. Further provided are diagnostic methods for diabetes and body lean mass. Furthermore, the invention relates to a method for identifying a treatment against diabetes and/or obesity.

### RELATED APPLICATIONS

This application is a divisional of patent application Ser. No. 13/003,314 filed Jan. 10, 2011, which is a national stage application (under 35 U.S.C. §371) of PCT/EP2009/059091, filed Jul. 15, 2009, which claims benefit of European application 08160396.1, filed Jul. 15, 2008. The entire content of each aforementioned application is hereby incorporated by reference in its entirety.

The present invention relates to the field of diagnostic measures. Specifically, it contemplates a method for assessing whether gastric bypass therapy was successful in a subject, a method of predicting whether gastric bypass therapy will be beneficial for a subject in need thereof, and a method of diagnosing whether a supportive therapy accompanying gastric bypass has beneficial effects on a subject in need thereof. Further provided are diagnostic methods for diabetes and body lean mass. Furthermore, the invention relates to a method for identifying a treatment against diabetes and/or obesity.

Obesity is characterized by the accumulation of excess body fat to an extent that health is adversely affected (i.e. via the development of comorbidities). Obesity is commonly defined as a body mass index (BMI, weight divided by height squared) of 30 kg/m2 or higher, while overweight is typically considered a BMI between 25-30. In 2005, the World Health Organization (WHO) estimated that approximately 1.6 billion people around the globe were overweight and 400 million adults were clinically defined as obese (who.int/en/). The WHO predicts that these numbers will increase to 2.3 billion overweight adults and 700 million obese adults by 2015. Inasmuch as society recognizes the important burden obesity has placed on the health care system, there are few means (not including dietary modifications and physical activity) by which to efficaciously improve the health status of overweight and obese individuals. Gastric bypass surgery has been demonstrated to be a very successful intervention by which to treat obesity and diabetes. Indeed, the most common form, Roux-en-Y, was performed on more than 120,000 people in the US in 2007 alone (Couzin 2008, Bypassing medicine to treat diabetes. Science 320, 438-440). The overall goal of gastric bypass surgery is the loss of body weight, specifically the loss of body fat mass, and the reversal of diabetes by improving insulin sensitivity. Gastric bypass may currently be the only cure for Type 2 diabetes and can normalize blood glucose levels in 80-100% of severely obese patients. Type 2 is the most prevalent form of diabetes. The prevalence of diagnosed and undiagnosed diabetes in the United States for all ages in 2007 was estimated to be 23.6 million people or 7.8 percent of the population. Of these 17.9 million people were diagnosed with diabetes and 5.7 million people had remained undiagnosed (National Diabetes Statistics 2007, US Department of Health and Human Services, diabetes.niddk.nih.gov/dm/pubs/statistics/). Diabetes it is up to 40 times more likely in those who are severely overweight.

Gastric bypass, a type of bariatric surgery, is a severe intervention increasingly applied in morbidly obese individuals in order to improve obesity and diabetes, while reducing the risk for comorbidities (Moo & Rubino 2008. Gastrointestinal surgery as treatment for type 2 diabetes. Curr Opin Endocrinol Diabetes Obes. 15: 153-8. Gumbs et al. 2005. Changes in insulin resistance following bariatric surgery: role of caloric restriction and weight loss. Obes Surg. 15: 462-73). The Consensus Panel of the National Institutes of Health (NIH) recommended criteria for the consideration of bariatric surgery. People who have a BMI of 40 or higher or people with a BMI of 35 or higher with one or more related comorbidities may be recommended to undergo gastric bypass if other weight loss therapies remain unsuccessful. The typical weight loss achieved with open or laparoscopic Roux-en-Y gastric bypass is 50-80% of excess body weight; however, considerable inter-individual differences can be expected. Gastric bypass surgery has been widely performed on morbidly obese patients and has been shown to reduce the death rate from all causes by up to 40% (Adams et al 2007. Long-term mortality after gastric bypass surgery. N. Engl. J. Med. 357, 753-61).

In most patients gastric bypass results in metabolic changes and normalizes blood glucose and insulin levels, insulin sensitivity and hormonal responses; however, the metabolic improvements related to diabetic endpoints represent but a fraction of the many alterations observed. For example, inflammatory markers such as C-reactive protein (CRP), serum amyloid A (SAA), IL-6, IL-18, sialic acid and TNF-α all decrease following bypass surgery, while adiponectin increases (Holdstock et al. 2005. CRP reduction following gastric bypass surgery is most pronounced in insulin-sensitive subjects. Int J Obes (Lond) 29: 1275-80. Catalan et al. 2007. Proinflammatory cytokines in obesity: impact of type 2 diabetes mellitus and gastric bypass. Obes Surg 17: 1464-1474. Vilarrasa et al. 2007. Effect of weight loss induced by gastric bypass on proinflammatory interleukin-18, soluble tumour necrosis factor-alpha receptors, C-reactive protein and adiponectin in morbidly obese patients. Clin Endocrinol (Oxf) 67: 679-686). Furthermore, lipid metabolism is modified following gastric bypass surgery and has been found to be correlated with the physical length of the bypass, e.g. free fatty acids and beta-hydroxybutrate levels were increased (Johansson et al. 2008. Lipid Mobilization Following Roux-en-Y Gastric Bypass Examined by Magnetic Resonance Imaging and Spectroscopy. Obes Surg. 2008 Apr. 8.). However, the physiological and mechanistic origins and consequences of these additional changes remain poorly understood.

Emerging evidence has clearly demonstrated the major role adipocytes play in both the storage of lipid and the secretion of hormones that influence feeding behavior, insulin sensitivity and immune function. While adipose gene expression (transcriptomic) studies have been performed in human patients to some extent, the global analysis of proteins (proteomics) and metabolites (metabolomics) has not yet been explored.

Severe obesity is associated with several comorbidities. Consequently gastric bypass patients need a comprehensive biochemical and clinical evaluation prior and after surgery and obtain multi-disciplinary support for optimum outcome. Usual bioclinical assessments include physiological measurements (body weight, body mass index, body fat mass and lean body mass), blood biochemistry (plasma triacylglycerols, cholesterol, lipoproteins, glucose, insulin, albumin, vitamin and mineral status), assessment of diet and medication, and controlling for the risk of specific comorbidities. A number of comorbidities have been demonstrated to improve following gastric bypass surgery.

Diabetic patients usually experience a partial, if not total, remission of diabetes as a result of gastric bypass, as indicated by normalized fasting blood glucose and insulin levels, and improved insulin sensitivity without medication. The effect is independent of weight loss and occurs within days after surgery. The pattern of secretion of gastrointestinal hormones is changed by gastric bypass and removal of the duodenum and proximal jejunum, the upper part of the small intestine. The surgery affects release and plasma concentrations of gastric hormones glucagon-like peptide-1 (GLP-1), ghrelin, and peptide YY (Ie Roux et al. 2006. Gut hormone profiles following bariatric surgery favor an anorectic state, facilitate weight loss, and improve metabolic parameters. Ann Surg. 243, 108-14; Rodieux et al. 2008. Effects of gastric bypass and gastric banding on glucose kinetics and gut hormone release. Obesity (Silver Spring). 16: 298-305; Reinehr et al. 2007. Peptide YY and glucagon-like peptide-1 in morbidly obese patients before and after surgically induced weight loss. Obes Surg. 17: 1571-7.). After gastric bypass, improved availability and efficacy of GLP-1, glucose-dependent insulinotropic polypeptide and incretin are in part responsible for the improved diabetic state (Laferrère et al. 2008, Effect of weight loss by gastric bypass surgery versus hypocaloric diet on glucose and incretin levels in patients with type 2 diabetes. J Clin Endocrinol Metab. 2008 Apr. 22). Diabetes is associated with a range of other diseases, including cardiovascular disease, kidney failure, blindness and nerve damage that can necessitate amputations of extremities.

Other comorbidities that are improved following gastric bypass surgery include essential hypertension, gastroesophageal reflux disease, venous thromboembolic disease, nonalcoholic fatty liver disease (nonalcoholic hepatic steatosis) and chronic inflammation of the liver (steatohepatitis), degeneration affecting the cartilaginous disks and the weight bearing joints, or osteoarthritis, affecting the hips, knees, ankles and feet. For example, hepatic steatose and fibrosis improved markedly in the 2 years following gastric bypass (assessed in NAFLD patients) (Furuya et al. 2007. Effects of bariatric surgery on nonalcoholic fatty liver disease: preliminary findings after 2 years. J Gastroenterol Hepatol. 22:510-4.).

Successful reversal of obesity is achieved by losing body fat and increasing the fraction of lean body mass. Imaging methods exist to measure body mass composition, where the most common is dual energy X-ray absorptiometry (DEXA or DXA) (Cunningham 1991. Body composition as a determinant of energy expenditure: a synthetic review and a proposed general prediction equation. Am J Clin Nutr, 54: 963-9). The technique is based on two types of X-ray body scans, one that detects all tissues and another that detects non-fat tissues, where body fat and lean mass are calculated from the difference in scans. Generally DEXA is considered the “gold standard” for measuring body fat and lean mass because of its general ease to use and its high degree of accuracy; however, DEXA instrumentation is expensive and generally not suitable for subjects weighing more than 150 kgs (postgradmed.com/issues/2003/12_{—}03/1bray.shtml). Nevertheless, it has been observed that weight loss after gastric bypass specifically jeopardizes skeletal muscle mass. Its maintenance during intentional weight loss can be achieved by a combination of physical exercise, a high fraction of dietary protein and other lifestyle adjustments. The most successful therapy depends on the individual predisposition. Gastric bypass success depends in part on monitoring lean body mass retention that can be assessed by the percentage of fat mass of the patient.

Nutrient deficiencies need to be prevented after gastric bypass intervention. Post-surgery patients feel fullness after ingesting only a small volume of food, followed soon thereafter by a sense of satiety and loss of appetite. Post surgery the total food intake is markedly reduced and bears the risk of lacking sufficient supply of essential micronutrients such as vitamins, minerals, carotenoids, essential fatty acids and protein (Gasteyger et al. 2008. Nutritional deficiencies after Roux-en-Y gastric bypass for morbid obesity often cannot be prevented by standard multivitamin supplementation. Am J Clin Nutr. 2008, 87: 1128-33). One reason for reduced nutrient availability is the reduced intestinal surface that leads to loss of nutrients which cannot be adequately absorbed from the diet. The reduced food intake demands that the patient follow the physician or dietician's instructions for food consumption and dietary supplementation of micronutrients and protein.

Gastric bypass surgery results in reduced energy intake and thus an energy restricted (caloric restricted) status (Ingram et al. 2006. Calorie restriction mimetics: an emerging research field. Aging Cell, 5: 97-108). The post surgery voluntarily food restriction in gastric bypass patients is one of the rare physiological conditions, in which humans experience a sustained energy deficient condition and health benefits. Caloric restriction (CR) aims to improve health and prolong the healthy lifespan when sufficient quantities of essential nutrients are provided. All animal models (primates, rats, mice, Drosophila, C. elegans and others) in which the effects of CR have been examined have demonstrated an extension of lifespan with an improved health status. In humans CR was reported to lower plasma lipids, fasting plasma glucose and insulin and blood pressure. Caloric restriction results in better protection from oxidative stress, reduced glycation of macromolecules, reduced DNA damage and increased repair, reduced inflammation and autoimmunity, increased mitochondrial metabolic efficiency to protect plasma membrane, reduced damage to cellular components (lysosomes, peroxisomes), enhanced maintenance of age-related patterns of gene expression and enhanced protection against stress (Ingram et al. 2006). Exact monitoring of the energy restricted state is inevitable to avoid undesirable effects such as anemia, muscle wasting, weakness, dizziness, fatigue, nausea, diarrhea, constipation, gallstones, irritability and depression in gastric bypass patients.

The mechanisms for beneficial effects of controlled restriction of dietary energy are poorly understood. The extended healthy lifespan associated with CR may be reached through hormesis, the chronic low-intensity biological stress imposed on mitochondria that elicits a defense response that helps protect against causes of aging. CR also improves insulin signaling. In mammals the SIRT1 gene is turned on by a CR diet or by dietary components such as resveratrol and protects cells from stress-induced death (Guarente 2008, Mitochondria—a nexus for aging, calorie restriction, and sirtuins? Cell. 132: 171-6).

The energy deficient state induced after gastric bypass surgery, however, may also provide a good model for geriatric anorexia and anorexia associated with diseases such as HIV-Aids and cancer. New understanding and the development of methods to ensure a more efficient supply of nutrients to these patients are urgently needed.

Taken together gastric bypass has been demonstrated to be very efficient in reducing body weight and diabetic symptoms in severely obese subjects. Due to the complexity of obesity diseases there is currently insufficient understanding of the patient's exact physiology prior to and after gastric bypass intervention. The obese and diabetic populations will greatly benefit from the better understanding of the physiological effects of gastric bypass surgery, as this improved knowledge state will ameliorate decision making for patient care. Furthermore, this new knowledge may lead towards the development of gastric bypass surgery “mimetics,” which capitalize on the health and aging retardation benefits of caloric restriction. Finally, an improved understanding of the metabolic effects associated with gastric bypass surgery will help to develop effective interventions that combat anorectic wasting diseases.

Accordingly, the technical problem underlying the present invention could be seen as the provision of means and methods for complying with the aforementioned needs. The technical problem is solved by the embodiments characterized in the claims and described herein below.

The present invention relates to a method of assessing whether gastric bypass therapy was successful in a subject comprising:
a) determining the amount of at least one biomarker selected from the group of biomarkers shown in any one of Tables 1A, 1B, 3 and 5 in a sample of said subject; and
b) comparing said amount to a reference, whereby it is to be assessed whether gastric bypass therapy was successful.

The method as referred to in accordance with the present invention includes a method which essentially consists of the aforementioned steps or a method which includes further steps. However, it is to be understood that the method, in a preferred embodiment, is a method carried out ex vivo, i.e. not practiced on the human or animal body. The method, preferably, can be assisted by automation.

The phrase “assessing whether gastric bypass therapy was successful” as used herein refers to determining whether a subject which has been treated by a gastric bypass therapy has a benefit from the said therapy, or not. Said benefit, preferably, is an amelioration of the diabetes and/or obesity symptoms or any other improvement with respect to the said medical conditions. Preferably, success with respect to diabetes is accompanied by an increase in insulin sensitivity (i.e. reduced insulin resistance), success with respect to obesity by a reduced % body fat mass. Preferably, the amelioration will be amelioration to a statistically significant extent. As will be understood by those skilled in the art, such an assessment, although preferred to be, may usually not be correct for 100% of the investigated subjects. The term, however, requires that a statistically significant portion of subjects can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90%, at least 95%. The p-values are, preferably, 0.2, 0.1, 0.05.

Assessing according to the present invention includes diagnosing, monitoring or confirming the success of a gastric bypass therapy. Moreover, the term also includes predicting whether the long-term outcome of a gastric bypass therapy will be successful, e.g., at an early stage after the application of the therapy when an amelioration of the symptoms or other improvements of the medical conditions referred to above are not yet clinically detectable.

The term “gastric bypass therapy” as used herein relates to measures of bariatric surgery whereby a small pouch is created from the upper stomach. The small intestine is then rearranged. The proximal part of the small intestine is bypassed and a distal part is directly connected to the gastric pouch. Gastric bypass therapies comprise open and laparoscopic Roux en-Y procedures. The surgery techniques are well known to the clinician and are described in standard text books of surgery. As a consequence of the gastric bypass on the physiology of a subject, obesity can be treated. Moreover, it has been found that a gastric bypass also ameliorates diabetes in a subject. This is of particular relevance, since a significant portion of subjects suffering from obesity also exhibit diabetes. Diabetes as meant in accordance with the aforementioned method of the invention refers to diabetes mellitus and, preferably, to type 2 diabetes mellitus. Obesity is a medical condition wherein the energy reserve stored in the fatty tissue of a subject exceeds healthy limits. It is preferably accompanied by a body mass index (weight divided by height squared) of at least 30 kg/m².

The term “biomarker” as used herein refers to a molecular species which serves as an indicator for a medical condition or effect as referred to in this specification. Said molecular species can be a metabolite itself which is found in a sample of a subject. Moreover, the biomarker may also be a molecular species which is derived from said metabolite. In such a case, the actual metabolite will be chemically modified in the sample or during the determination process and, as a result of said modification, a chemically different molecular species, i.e. the analyte, will be the determined molecular species. It is to be understood that in such a case, the analyte represents the actual metabolite and has the same potential as an indicator for the respective medical condition.

Preferably, at least one metabolite of the aforementioned group of biomarkers is to be determined in the method of the present invention. However, more preferably, a group of biomarkers will be determined in order to strengthen specificity and/or sensitivity of the assessment. Such a group, preferably, comprises at least 2, at least 3, at least 4, at least 5, at least 10 or up to all of the said biomarkers. In addition to the specific biomarkers recited in the specification, other biomarkers may be, preferably, determined as well in the methods of the present invention.

A metabolite as used herein refers to at least one molecule of a specific metabolite up to a plurality of molecules of the said specific metabolite. It is to be understood further that a group of metabolites means a plurality of chemically different molecules wherein for each metabolite at least one molecule up to a plurality of molecules may be present. A metabolite in accordance with the present invention encompasses all classes of organic or inorganic chemical compounds including those being comprised by biological material such as organisms. Preferably, the metabolite in accordance with the present invention is a small molecule compound. More preferably, in case a plurality of metabolites is envisaged, said plurality of metabolites representing a metabolome, i.e. the collection of metabolites being comprised by an organism, an organ, a tissue, a body fluid or a cell at a specific time and under specific conditions.

The metabolites are small molecule compounds, such as substrates for enzymes of metabolic pathways, intermediates of such pathways or the products obtained by a metabolic pathway. Metabolic pathways are well known in the art and may vary between species. Preferably, said pathways include at least citric acid cycle, respiratory chain, photosynthesis, photorespiration, glycolysis, gluconeogenesis, hexose monophosphate pathway, oxidative pentose phosphate pathway, production and n-oxidation of fatty acids, urea cycle, amino acid biosynthesis pathways, protein degradation pathways such as proteasomal degradation, amino acid degrading pathways, biosynthesis or degradation of: lipids, polyketides (including e.g. flavonoids and isoflavonoids), isoprenoids (including eg. terpenes, sterols, steroids, carotenoids, xanthophylls), carbohydrates, phenylpropanoids and derivatives, alcaloids, benzenoids, indoles, indolesulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs. Accordingly, small molecule compound metabolites are preferably composed of the following classes of compounds: alcohols, alkanes, alkenes, alkines, aromatic compounds, ketones, aldehydes, carboxylic acids, esters, amines, imines, amides, cyanides, amino acids, peptides, thiols, thioesters, phosphate esters, sulfate esters, thioethers, sulfoxides, ethers, or combinations or derivatives of the aforementioned compounds. The small molecules among the metabolites may be primary metabolites which are required for normal cellular function, organ function or animal growth, development or health. Moreover, small molecule metabolites further comprise secondary metabolites having essential ecological function, e.g. metabolites which allow an organism to adapt to its environment. Furthermore, metabolites are not limited to said primary and secondary metabolites and further encompass artificial small molecule compounds. Said artificial small molecule compounds are derived from exogenously provided small molecules which are administered or taken up by an organism but are not primary or secondary metabolites as defined above. For instance, artificial small molecule compounds may be metabolic products obtained from drugs by metabolic pathways of the animal. Moreover, metabolites further include peptides, oligopeptides, polypeptides, oligonucleotides and polynucleotides, such as RNA or DNA. More preferably, a metabolite has a molecular weight of 50 Da (Dalton) to 30,000 Da, most preferably less than 30,000 Da, less than 20,000 Da, less than 15,000 Da, less than 10,000 Da, less than 8,000 Da, less than 7,000 Da, less than 6,000 Da, less than 5,000 Da, less than 4,000 Da, less than 3,000 Da, less than 2,000 Da, less than 1,000 Da, less than 500 Da, less than 300 Da, less than 200 Da, less than 100 Da. Preferably, a metabolite has, however, a molecular weight of at least 50 Da. Most preferably, a metabolite in accordance with the present invention has a molecular weight of 50 Da up to 1,500 Da.

Further, as specified below in detail, some biomarkers are particularly preferred for assessing whether gastric bypass therapy was successful with respect to diabetes while other biomarkers are particularly preferred for predicting or diagnosing whether gastric bypass therapy was successful with respect to obesity.

Thus, in a preferred embodiment of the method of the present invention, said assessing comprises assessing whether gastric bypass therapy was successful with respect to diabetes based on the comparison of at least one biomarker selected from the group of biomarkers shown in Table 2 and 3.

Moreover, in another preferred embodiment of the method of the present invention, said assessing comprises assessing whether gastric bypass therapy was successful with respect to obesity based on the comparison of at least one biomarker selected from the group of biomarkers shown in Tables 4 and 5.

More preferably, the present invention also comprises assessing whether gastric bypass therapy was successful with respect to diabetes and obesity based on the comparison of at least one biomarker selected from the group of biomarkers shown in Table 1A and/or 1B.

The term “sample” as used herein refers to samples from body fluids, preferably, blood, plasma, serum, saliva, urine or cerebrospinal fluid, or samples derived, e.g., by biopsy, from cells, tissues or organs. More preferably, the sample is a blood, plasma or serum sample, most preferably, a plasma sample. Biological samples can be derived from a subject as specified elsewhere herein. Techniques for obtaining the aforementioned different types of biological samples are well known in the art. For example, blood samples may be obtained by blood taking while tissue or organ samples are to be obtained, e.g., by biopsy.

The aforementioned samples are, preferably, pre-treated before they are used for the method of the present invention. As described in more detail below, said pre-treatment may include treatments required to release or separate the compounds or to remove excessive material or waste. Suitable techniques comprise centrifugation, extraction, fractioning, ultrafiltration, protein precipitation followed by filtration and purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivative the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

The sample according to the aforementioned method has been taken from the subject directly before or after application of the gastric therapy. Preferably, the sample can be taken prior or 3 or 6 month after gastric bypass therapy.

The term “subject” as used herein relates to animals and, preferably, to mammals. More preferably, the subject is a primate and, most preferably, a human.

The term “determining the amount” as used herein refers to determining at least one characteristic feature of a biomarker to be determined by the method of the present invention in the sample. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemoluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the said at least one biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z information, as well as an intensity value being related to the abundance of the said biomarker (i.e. its amount) in the sample.

As discussed before, each biomarker comprised by a sample may be, preferably, determined in accordance with the present invention quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present is enlarged or diminished with respect to a second sample comprising said biomarker in a second amount. In a preferred embodiment said second sample comprising said biomarker shall be a calculated reference as specified elsewhere herein. Quantitatively analysing a biomarker, thus, also includes what is sometimes referred to as semi-quantitative analysis of a biomarker.

Moreover, determining as used in the method of the present invention, preferably, includes using a compound separation step prior to the analysis step referred to before. Preferably, said compound separation step yields a time resolved separation of the metabolites comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the method of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen, Journal of Chromatography A, 703, 1995: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

Moreover, the at least one biomarker can also be determined by a specific chemical or biological assay. Said assay shall comprise means which allow to specifically detect the at least one biomarker in the sample. Preferably, said means are capable of specifically recognizing the chemical structure of the biomarker or are capable of specifically identifying the biomarker based on its capability to react with other compounds or its capability to elicit a response in a biological read out system (e.g., induction of a reporter gene). Means which are capable of specifically recognizing the chemical structure of a biomarker are, preferably, antibodies or other proteins which specifically interact with chemical structures, such as receptors or enzymes. Specific antibodies, for instance, may be obtained using the biomarker as antigen by methods well known in the art. Antibodies as referred to herein include both polyclonal and monoclonal antibodies, as well as fragments thereof, such as Fv, Fab and F(ab)₂ fragments that are capable of binding the antigen or hapten. The present invention also includes humanized hybrid antibodies wherein amino acid sequences of a non-human donor antibody exhibiting a desired antigen-specificity are combined with sequences of a human acceptor antibody. Moreover, encompassed are single chain antibodies. The donor sequences will usually include at least the antigen-binding amino acid residues of the donor but may comprise other structurally and/or functionally relevant amino acid residues of the donor antibody as well. Such hybrids can be prepared by several methods well known in the art. Suitable proteins which are capable of specifically recognizing the biomarker are, preferably, enzymes which are involved in the metabolic conversion of the said biomarker. Said enzymes may either use the biomarker as a substrate or may convert a substrate into the biomarker. Moreover, said antibodies may be used as a basis to generate oligopeptides which specifically recognize the biomarker. These oligopeptides shall, for example, comprise the enzyme's binding domains or pockets for the said biomarker. Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Moreover, the biomarker may also be determined based on its capability to react with other compounds, i.e. by a specific chemical reaction. Further, the biomarker may be determined in a sample due to its capability to elicit a response in a biological read out system. The biological response shall be detected as read out indicating the presence and/or the amount of the biomarker comprised by the sample. The biological response may be, e.g., the induction of gene expression or a phenotypic response of a cell or an organism. In a preferred embodiment the determination of the least one biomarker is a quantitative process, e.g., allowing also the determination of the amount of the at least one biomarker in the sample

As described above, said determining of the at least one biomarker comprises mass spectrometry (MS). Mass spectrometry as used herein encompasses all techniques which allow for the determination of the molecular weight (i.e. the mass) or a mass variable corresponding to a compound, i.e. a biomarker, to be determined in accordance with the present invention. Preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS, ICP-MS, Py-MS, TOF or any combined approaches using the aforementioned techniques. How to apply these techniques is well known to the person skilled in the art. Moreover, suitable devices are commercially available. More preferably, mass spectrometry as used herein relates to LC-MS and/or GC-MS, i.e. to mass spectrometry being operatively linked to a prior chromatographic separation step. More preferably, mass spectrometry as used herein encompasses quadrupole MS. Most preferably, said quadrupole MS is carried out as follows: a) selection of a mass/charge quotient (m/z) of an ion created by ionisation in a first analytical quadrupole of the mass spectrometer, b) fragmentation of the ion selected in step a) by applying an acceleration voltage in an additional subsequent quadrupole which is filled with a collision gas and acts as a collision chamber, c) selection of a mass/charge quotient of an ion created by the fragmentation process in step b) in an additional subsequent quadrupole, whereby steps a) to c) of the method are carried out at least once and analysis of the mass/charge quotient of all the ions present in the mixture of substances as a re-suit of the ionisation process, whereby the quadrupole is filled with collision gas but no acceleration voltage is applied during the analysis. Details on said most preferred mass spectrometry to be used in accordance with the present invention can be found in WO 03/073464.

More preferably, said mass spectrometry is liquid chromatography (LC) MS and/or gas chromatography (GC) MS.

Liquid chromatography as used herein refers to all techniques which allow for separation of compounds (i.e. metabolites) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. Gas chromatography as applied in accordance with the present invention, in principle, operates comparable to liquid chromatography. However, rather than having the compounds (i.e. metabolites) in a liquid mobile phase which is passed through the stationary phase, the compounds will be present in a gaseous volume. The compounds pass the column which may contain solid support materials as stationary phase or the walls of which may serve as or are coated with the stationary phase. Again, each compound has a specific time which is required for passing through the column. Moreover, in the case of gas chromatography it is preferably envisaged that the compounds are derivatised prior to gas chromatography. Suitable techniques for derivatisation are well known in the art. Preferably, derivatisation in accordance with the present invention relates to methoxymation and trimethylsilylation of, preferably, polar compounds and transmethylation, methoxymation and trimethylsilylation of, preferably, non-polar (i.e. lipophilic) compounds.

The term “reference” refers to values of characteristic features of each of the biomarker which can be correlated to the medical conditions or effects referred to herein. Preferably, a reference is a threshold amount for a biomarker whereby amounts found in a sample to be investigated which are higher than or identical to the threshold are indicative for the presence of a medical condition while those being lower are indicative for the absence of the medical condition. It will be understood that also preferably, a reference may be a threshold amount for a biomarker whereby amounts found in a sample to be investigated which are lower or identical than the threshold are indicative for the presence of a medical condition while those being higher are indicative for the absence of the medical condition.

In accordance with the aforementioned method of the present invention, a reference is, preferably a reference amount obtained from a sample from a subject known to have been successfully treated by a gastric bypass therapy. In such a case, an amount for the at least one biomarker found in the test sample being identical or similar is indicative for a successful treatment by the gastric bypass therapy or from a healthy subject with respect to obesity and/or diabetes. Moreover, the reference, also preferably, could be a calculated reference, most preferably the average or median, for the relative or absolute amount of the at least one biomarker of a population of individuals comprising the subject to be investigated. The absolute or relative amounts of the at least one biomarker of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 or 10,000 subjects. It is to be understood that the subject to be assessed by the method of the present invention and the subjects of the said plurality of subjects are of the same species. Also in the latter case, an amount of the at least one biomarker in the test sample being identical or similar to the reference is indicative for a successful treatment by the gastric bypass therapy. The amounts of the test sample and the reference amounts are identical, if the values for the characteristic features and, in the case of quantitative determination, the intensity values are identical. Said amounts are similar, if the values of the characteristic features are identical but the intensity values are different. Such a difference is, preferably, not significant and shall be characterized in that the values for the intensity are within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference value, preferably, the 50^{th}, 60^{th}, 70^{th}, 80^{th}, 90^{th} or 95^{th} percentile of the reference value.

Alternatively, but nevertheless also preferred, the reference amounts may be obtained from sample of a subject known not to have been successfully treated by a gastric bypass therapy. In said case, an amount in the test sample for the at least one biomarker which differs from the reference is indicative for a gastric bypass therapy being successful. Moreover, reference is also preferably the amount of the at least one biomarker which is to be determined in a sample of the subject prior to applying the gastric bypass therapy, i.e. the subject when suffering from obesity and/or diabetes. In such a case, a difference in the amount of the at least one biomarker between the sample obtained prior (i.e. the reference) and after the application of the treatment (i.e. the test sample amount) will be indicative for an effective treatment to be identified by the aforementioned method of the invention. Preferably, the observed difference shall be statistically significant. A difference in the relative or absolute amount is, preferably, significant outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1^{st} and 99^{th} percentile of the reference value. Preferred changes and fold-regulations are described in the accompanying Tables 1A, 1B, 3 and 5 as well as in the Examples.

Preferably, the reference, i.e. values for at least one characteristic features of the at least one biomarker, will be stored in a suitable data storage medium such as a database and are, thus, also available for future assessments.

The term “comparing” refers to determining whether the determined amount of a biomarker is identical or similar to a reference or differs therefrom. Preferably, a biomarker is deemed to differ from a reference if the observed difference is statistically significant which can be determined by statistical techniques referred to elsewhere in this description. Specifically, the amount of the test sample and the reference are identical, if the values for the characteristic features and, in the case of quantitative determination, the intensity values are identical. Said results are similar, if the values of the characteristic features are identical but the intensity values are different. Such a difference is, preferably, not significant and shall be characterized in that the values for the intensity are within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference value, preferably, the 50^{th}, 60^{th}, 70^{th}, 80^{th}, 90^{th} or 95^{th} percentile of the reference value. Based on the comparison referred to above, a subject can be allocated to the group of subject which were successfully treated by a gastric bypass therapy, or not.

For the specific biomarkers referred to in this specification, preferred values for the changes in the relative amounts (i.e. “fold”-changes) or the kind of change (i.e. “up”- or “down”-regulation resulting in a higher or lower relative and/or absolute amount) are indicated in the following Tables 1 to 5 and in the Examples below. If it is indicated in said table that a given biomarker is “up-regulated” in a subject, the relative and/or absolute amount will be increased, if it is “down-regulated”, the relative and/or absolute amount of the biomarker will be decreased. Moreover, the “fold”-change indicates the degree of increase or decrease, e.g., a 2-fold increase means that the amount is twice the amount of the biomarker compared to the reference.

The comparison is, preferably, assisted by automation. For example, a suitable computer program comprising algorithms for the comparison of two different data sets (e.g., data sets comprising the values of the characteristic feature(s)) may be used. Such computer programs and algorithm are well known in the art. Notwithstanding the above, a comparison can also be carried out manually.

Advantageously, it has been found in the study underlying the present invention that the amounts of the specific biomarkers referred to above are indicators for the success of a gastric bypass therapy. Accordingly, the at least one biomarker as specified above in a sample can, in principle, be used for assessing whether a gastric bypass therapy was successful for a subject in need thereof. Moreover, the biomarkers even allow further conclusions in particular assessing the success of a gastric bypass therapy with respect to diabetes and/or obesity. Thanks to the present invention, the effectiveness of bariatric surgery and, in particular, gastric bypass therapy can be assessed on reliable and efficient outcome parameters, i.e. the biomarkers referred to above. Moreover, the biomarkers also allow prediction of the long-term outcome of the therapy with respect to diabetes and/or obesity. This is particularly helpful for a individual risk stratification of future adverse events or reoccurrence of the diseases for a subject and, consequently, for individual recommendations with respect to further diagnostic and therapeutic measures for a subject. Moreover, the findings underlying the present invention will also facilitate the development of further bariatric or drug based therapies against diabetes and/or obesity as set forth in detail below.

The definitions and explanations of the terms made above apply mutatis mutandis for the following embodiments of the present invention except specified otherwise herein below.

The present invention, further, relates to a method of predicting whether gastric bypass therapy will be beneficial for a subject in need thereof comprising
a) determining the amount of at least one biomarker selected from the group of the biomarkers shown in Tables 6 and 7 in a sample of said subject; andunt to a reference, whereby it is to be predicted whether gastric bypass therapy will be beneficial.

The term “predicting” as used herein refers to determining the probability according to which a subject will benefit from a future gastric bypass therapy. It will be understood that such a prediction will not necessarily be correct for all (100%) of the investigated subjects. However, it is envisaged that the prediction will be correct for a statistically significant portion of subjects of a population of subjects (e.g., the subjects of a cohort study). Whether a portion is statistically significant can be determined by statistical techniques set forth elsewhere herein.

Moreover, it is to be understood that gastric bypass therapy will be beneficial for a subject if the gastric bypass therapy will be successful as described elsewhere herein at least with a likelihood of success being greater than the likelihood of failure or the likelihood for developing adverse complications due to the gastric bypass therapy.

It will be understood that a subject in need for a gastric bypass therapy as meant herein is, preferably, a subject suffering from obesity, preferably, in combination with diabetes. Moreover, in accordance with the aforementioned method, the said sample has been obtained from a subject which has not been subjected to a gastric bypass therapy, yet.

Further, a reference in accordance with the aforementioned method is, preferably, a reference amount for the at least one biomarker determined in a sample of a subject known to be successfully treated by a gastric bypass therapy wherein the sample was obtained prior to the said therapy. In such a case, an amount for the at least one biomarker determined in the investigated sample being identical or similar to the reference amount is indicative for a subject for which gastric bypass therapy will be beneficial. Alternatively, but nevertheless also preferred, the reference can be a reference amount for the at least one biomarker determined in a sample of a subject known be treated by a gastric bypass therapy without success wherein the sample was obtained prior to the said therapy. In said case, an amount for the at least one biomarker determined in the investigated sample being different from the reference amount is indicative for a subject for which gastric bypass therapy will be beneficial while an identical or similar amount for the at least one biomarker indicates that the subject will not benefit from gastric bypass therapy. Preferred changes in the regulation of the at least one biomarker are shown in the Tables 6 and 7 and Examples, below.

Advantageously, the aforementioned method of the present invention allows for risk assessment of gastric bypass therapies. Specifically, based on the result of this method, subjects can be excluded from the therapy in case they are at risk of having no benefit from the therapy. Therefore, adverse complications can be avoided and, furthermore, the gastric bypass therapies can be applied more cost effective. The biomarkers referred to in accordance with the method comprised by a sample have, in principle, be found to be useful for predicting whether gastric bypass therapy will be beneficial for a subject in need thereof.

Also contemplated by the present invention is a method of determining whether a supportive therapy accompanying gastric bypass has beneficial effects on a subject in need thereof comprising:
a) determining the amount of at least one biomarker selected from the group of biomarkers shown in Table 8 in a sample of said subject; and
b) comparing said amount to a reference, it is to be determined whether the supplement diet has beneficial effects.

The term “supportive therapy” as used herein refers to therapeutic measures which are applied to a subject in order to increase the likelihood of success for a gastric bypass therapy. The term includes drug-based or physical therapies as well as recommendations on nutrition or supplementation. Preferably, said supportive therapy is selected from the group consisting of: nutritional therapy, a dietary supplement, a drug and combinations thereof.

Such a supportive therapy is deemed to have beneficial effects on a subject if the supportive therapy increases the likelihood of success for the gastric bypass therapy, reduces the risk for developing adverse complications or at least improves the overall well being of the subject.

Preferred values for the changes with respect to the reference of the at least one biomarker are to be found in the accompanying Table 8 and Examples, below. The preferred values indicated deficiency of the subject after gastric bypass therapy in respect to certain metabolites in comparison to the reference. Preferably, the supportive therapy is a supplementation of the metabolite which serves as a biomarker in the aforementioned method.

In accordance with the present invention, it has been found, in principle, that the aforementioned metabolites in a sample of a subject can be used for diagnosing whether a supportive therapy accompanying gastric bypass has beneficial effects.

Thanks to the aforementioned method of the present invention, it can be readily and reliably determined whether a supportive therapy is beneficial for a subject having been treated by a gastric bypass therapy. The method, thus, allows refraining from supportive therapies which have no beneficial effects for the subject and to, rather, focus on those which do have beneficial effects.

The present invention also relates to a method of diagnosing diabetes in a subject comprising:
a) determining the amount of at least one biomarker selected from the group of biomarkers shown in Tables 9 and 10 or a combination of biomarkers as recited in Table 15 in a sample of said subject; and
b) comparing said amount to a reference, whereby diabetes is to be diagnosed.

Diagnosing as used herein refers to assessing the probability according to which a subject is suffering from a disease. As will be understood by those skilled in the art, such an assessment, although preferred to be, may usually not be correct for 100% of the subjects to be diagnosed. The term, however, requires that a statistically significant portion of subjects can be identified as suffering from the disease or as having a predisposition therefore. Whether a portion is statistically significant can be determined without further ado by the person skilled in the art using various well known statistic evaluation tools set forth elsewhere in this specification.

Diagnosing according to the present invention includes monitoring, confirmation, and classification of the relevant disease or its symptoms. Monitoring relates to keeping track of an already diagnosed disease, or a complication, e.g. to analyze the progression or remission of the disease, the influence of a particular treatment on the progression of disease or complications arising during the disease period or after successful treatment of the disease. Confirmation relates to the strengthening or substantiating a diagnosis already performed using other indicators or markers. Classification relates to allocating the diagnosis according to the strength or kind of symptoms into different classes, e.g. the diabetes types as set forth elsewhere in the description.

Some of the aforementioned biomarkers are, preferably, indicators of the presence, absence or strength of the disease, i.e. conventional diagnostic indicators (see, preferably, Table 9) whereas other are indicators for progression or remission of the disease (see, preferably, Table 10).

Preferred combinations of biomarkers for diagnosing diabetes are the combinations 1 to 20 recited in Table 15, below.

The term “diabetes” or “diabetes mellitus” as used in accordance with the aforementioned method of the invention refers to disease conditions in which the glucose metabolism is impaired, in general. Said impairment results in hyperglycaemia. According to the World Health Organisation (WHO), diabetes can be subdivided into four classes. Type 1 diabetes is caused by a lack of insulin. Insulin is produced by the so called pancreatic islet cells. Said cells may be destroyed by an autoimmune reaction in Type 1 diabetes (Type 1a). Moreover, Type 1 diabetes also encompasses an idiopathic variant (Type 1b). Type 2 diabetes is caused by an insulin resistance. Type 3 diabetes, according to the current classification, comprises all other specific types of diabetes mellitus. For example, the beta cells may have genetic defects affecting insulin production, insulin resistance may be caused genetically or the pancreas as such may be destroyed or impaired. Moreover, hormone deregulation or drugs may also cause Type 3 diabetes. Type 4 diabetes may occur during pregnancy. Preferably, diabetes as used herein refers to diabetes Type 2. According to the German Society for Diabetes, diabetes is diagnosed either by a plasma glucose level being higher than 110 mg/dl in the fasting state or being higher than 220 mg/dl postprandial. Further preferred diagnostic techniques are disclosed elsewhere in this specification. Further symptoms of diabetes are well known in the art and are described in the standard text books of medicine, such as Stedman or Pschyrembl.

The term “reference” in the context of the aforementioned method of the present invention refers to reference amounts of the at least one biomarker which can be correlated to diabetes. Such reference amounts are, preferably, obtained from a sample from a subject known to suffer from diabetes. The reference amounts may be obtained by applying the method of the present invention. Alternatively, but nevertheless also preferred, the reference amounts may be obtained from sample from a subject known not to suffer from diabetes, i.e. a healthy subject with respect to diabetes and, more preferably, other diseases as well. Moreover, the reference, also preferably, could be a calculated reference, most preferably the average or median, for the relative or absolute amount of the at least one biomarker of a population of individuals comprising the subject to be investigated. The absolute or relative amounts of the at least one biomarker of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 or 10,000 subjects. It is to be understood that the subject to be diagnosed by the method of the present invention and the subjects of the said plurality of subjects are of the same species.

In case the reference is obtained from a subject or a group known to suffer from diabetes, the said disease can be diagnosed based on the degree of identity or similarity between the determined biomarker obtained from the test sample and the aforementioned reference, i.e. based on an identical or similar qualitative or quantitative composition with respect to the at least one biomarker.

In case the reference is obtained from a subject or a group known not to suffer from diabetes, the said disease can be diagnosed based on the differences between the determined amounts in the test sample and the aforementioned reference amounts, i.e. differences in the qualitative or quantitative composition with respect to the at least one biomarker. The same applies if a calculated reference as specified above is used. The difference may be an increase in the absolute or relative amount of the at least one biomarker (sometimes referred to as up-regulation; see also Examples) or a decrease in either of said amounts or the absence of a detectable amount of the at least one biomarker (sometimes referred to as down-regulation; see also Examples). For the specific biomarkers referred to in connection with the aforementioned method of the present invention, preferred values for the changes in the relative amounts (i.e. “fold”-changes) or the kind of change (i.e. “up”- or “down”-regulation resulting in a higher or lower relative and/or absolute amount) are indicated in Tables 9 to 10 below.

Thus, the method of the present invention in a preferred embodiment includes a reference that is derived from a subject or a group known to suffer from diabetes. Most preferably, identical or similar results for the test sample and the said reference (i.e. similar relative or absolute amounts of the at least one biomarker) are indicative for diabetes in that case. In another preferred embodiment of the method of the present invention, the reference is derived from a subject known not to suffer from diabetes or is a calculated reference, e.g, from a group of subjects known not to suffer from diabetes. Most preferably, the absence of the at least one biomarker or an amount which, preferably significantly, differs in the test sample in comparison to the reference (i.e. a significant difference in the absolute or relative amount is observed) is indicative for diabetes in such a case.

Advantageously, it has been found in the studies underlying the present invention that the biomarkers referred to in the context of the aforementioned method of the present invention are, particularly, useful in a sample of a subject for diagnosing diabetes, in general. Thanks to the present invention, diabetes can be more reliably and efficiently diagnosed and monitored and, consequently, diabetes care can be improved.

The present invention, furthermore, relates to a method of diagnosing body lean mass in a subject comprising:
a) determining the amount of at least one biomarker selected from the group of biomarkers as shown in Table 12 in a sample of said subject; and
b) comparing said amount to a reference, whereby the amount of body lean mass is to be diagnosed.

The term “body lean mass” as used herein refers to the body mass of a subject except the storage fat mass and the bone mass. The body lean mass is, preferably, expressed in percent of total body mass The body lean mass compared to the total body mass is an important indicator for diseases and disorders associated or caused by excessive body storage fat. Accordingly, a high body lean mass shall be preferably over a low body lean mass. A low body lean mass is, preferably, an indicator for an increased predisposition for diabetes and/or obesity. Moreover, the body lean mass change can be used as an indicator for determining whether a drug or exercise- or life style recommendations are effective for the overall health of a subject. The body lean mass is determined in the prior art by techniques which require specialized equipment such as underwater weighing (hydrostatic weighing), BOD POD (a computerized chamber), or dual-energy X-ray absorptiometry.

In accordance with the present invention, it has been found that the biomarkers referred to above are closely correlated to the body lean mass. Said correlation can be used for determining the body lean mass of a subject or to determine changes, i.e. to monitor a subject with respect to its body lean mass. If the body lean mass of a subject shall be determined, it will be required to calibrate the amount of the at least one biomarker with the amount of body lean mass. Based on, e.g., a calibration curve, the absolute amount of body lean mass can be calculated from the determined absolute amount of the at least one biomarker. Accordingly, a suitable reference in said case is, preferably, a calibrated value of the at least one biomarker or a calibration curve for the said at least one biomarker. Such a calibration can be done by the person skilled in the art without further ado. If relative changes are to be determined, the changes of the at least one biomarker in two or more samples of the subject can be determined wherein the said two or more samples have been obtained at different time points. Such time points are, preferably, separated by the onset of external stimuli such as the aforementioned drug administration or application of exercise or life style recommendations.

Thanks to the present invention, the body lean mass can be readily and reliably determined, especially as part of the clinical routine. Changes which affect a subjects risk for developing diseases and disorders associated or caused by excessive body storage fat, such as diabetes or obesity, can be closely monitored and the effectiveness of measures counteracting the said risk can be evaluated.

Moreover, the present invention encompasses a method of diagnosing the energy state of a subject comprising
a) determining the amount of at least one biomarker selected from the group of biomarkers shown in Table 11 in a sample of a subject; and
b) comparing said amount to a reference, whereby the energy state is to be identified.

The term “energy state” as used herein refers to the energy balance between energy uptake and energy expenditure. A negative energy state is characterized in that the energy expenditure exceeds the energy uptake. In other words, the subject burns more energy equivalents than it takes up. Consequently, the subject will not store energy equivalents in form of storage fat (i.e. having a negative energy state). Therefore, the risk for developing the above mentioned disorders or diseases accompanying a balanced or positive energy state will be significantly reduced. Moreover, the overall well being will be improved, the mortality rate will be reduced and aging process will be slowed down.

In accordance with the present invention, it has been found that the biomarkers referred to above are closely correlated to the energy state of a subject. Said correlation can be used for determining the absolute energy state of a subject or to determine changes, i.e. to monitor a subject with respect to its energy state. If the absolute energy state of a subject shall be determined, it will be required to calibrate the amount of the at least one biomarker with the energy state. Based on, e.g., a calibration curve, the absolute energy state can be calculated from the determined absolute amount of the at least one biomarker. Accordingly, a suitable reference in said case is, preferably, a calibrated value of the at least one biomarker or a calibration curve for the said at least one biomarker. Such a calibration can be done by the person skilled in the art without further ado. If relative changes are to be determined, the changes of the at least one biomarker in two or more samples of the subject can be determined wherein the said two or more samples have been obtained at different time points. Such time points are, preferably, separated by the onset of external stimuli such as the aforementioned drug administration or application of exercise or life style recommendations.

Thanks to the present invention, the energy state can be readily and reliably determined. As discussed for the previous methods of the invention, changes which affect a subjects risk for developing diseases and disorders associated or caused by excessive body storage fat, such as diabetes or obesity, can be closely monitored and the effectiveness of measures counteracting the said risk can be evaluated.

Moreover, the present invention relates to a method for identifying a treatment against diabetes and/or obesity comprising:
a) determining the amount of at least one biomarker selected from the group of biomarkers as shown in any one of Tables 1A, 1B, 3 and 5 in a sample of a subject to which a treatment suspected to be effective against diabetes and/or obesity has been applied; and
b) comparing said amount to a reference, whereby the treatment is to be identified.

The term “treatment” as used herein refers to therapeutic measures which are capable of treating or ameliorating diabetes and/or obesity or the symptoms accompanying these diseases. Preferably, said treatment is selected from the group consisting of: administration of drugs, nutritional diets, dietary supplements, surgery, bariatric surgery, supporting physical activity, life-style recommendations and combinations thereof.

It will be understood that the treatment as referred to in accordance with the aforementioned method will not be necessarily effective for all subjects to be treated. However, a treatment to be identified by the method shall at least be effective for a statistically significant portion of subjects of a population. Whether such a portion of subjects is statistically significant can be determined by techniques described elsewhere in this specification in detail.

Preferably, a treatment against diabetes is to be identified by at least one biomarker selected from the group as shown in Table 2 and 3 and/or a treatment against obesity is to be identified by at least one biomarker selected from the group as shown in Table 4 and 5.

Moreover, the term “subject” as used in accordance with the aforementioned method of the present invention refers to a subject which prior to the applied treatment suffered from diabetes and/or obesity.

The term “reference” in the context of the aforementioned method of the present invention refers to reference amounts of the at least one biomarker which are indicative for a successful treatment of diabetes and/or obesity. Such reference amounts are, preferably, obtained from a sample from a subject known to have been successfully treated. Preferably, said subject has been treated by a gastric bypass therapy as set forth elsewhere herein. The reference amounts may be obtained by applying the method of the present invention. Alternatively, but nevertheless also preferred, the reference amounts may be obtained from sample of a subject known not to suffer from diabetes and/or obesity, i.e. a healthy subject with respect to diabetes and/or obesity and, more preferably, other diseases as well. Moreover, the reference, also preferably, could be a calculated reference, most preferably the average or median, for the relative or absolute amount of the at least one biomarker of a population of individuals comprising the subject to be investigated. The absolute or relative amounts of the at least one biomarker of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 or 10,000 subjects. It is to be understood that the subject to be diagnosed by the method of the present invention and the subjects of the said plurality of subjects are of the same species. In case a the reference is obtained from a subject or a group known to have been successfully treated or a group known not to suffer from diabetes and/or obesity, the treatment can be identified based on the degree of identity or similarity between the determined biomarker obtained from the test sample and the aforementioned reference, i.e. based on an identical or similar qualitative or quantitative composition with respect to the at least one biomarker. The amounts of the test sample and the reference amounts are identical, if the values for the characteristic features and, in the case of quantitative determination, the intensity values are identical. Said amounts are similar, if the values of the characteristic features are identical but the intensity values are different. Such a difference is, preferably, not significant and shall be characterized in that the values for the intensity are within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference value, preferably, the 50^{th}, 60^{th}, 70^{th}, 80^{th}, 90^{th} or 95^{th} percentile of the reference value.

A “reference”, however, could also be the amount of the at least one biomarker which is to be determined in a sample of the subject prior to applying the treatment, i.e. the subject when suffering from obesity and/or diabetes. In such a case, a difference in the amount of the at least one biomarker between the sample obtained prior (i.e. the reference) and after the application of the treatment (i.e. the test sample amount) will be indicative for an effective treatment to be identified by the aforementioned method of the invention. Preferably, the observed difference shall be statistically significant as set forth elsewhere in this specification. Preferred changes and fold-regulations are described in the accompanying Tables 1A, 1B, 3 and 5 as well as in the Examples.

Advantageously, it has been found in the studies underlying the present invention that the biomarkers referred to in the context of the aforementioned method of the present invention are, particularly, useful for identifying a treatment against diabetes and/or obesity being effective. Thanks to the present invention, diabetes and obesity treatments can be reliably and efficiently identified. Moreover, it can be even assessed on an individual basis whether a treatment will be effective, or not.

The aforementioned methods for the determination of the at least one biomarker can be implemented into a device. A device as used herein shall comprise at least the aforementioned means. Moreover, the device, preferably, further comprises means for comparison and evaluation of the detected characteristic feature(s) of the at least one biomarker and, also preferably, the determined signal intensity. The means of the device are, preferably, operatively linked to each other. How to link the means in an operating manner will depend on the type of means included into the device. For example, where means for automatically qualitatively or quantitatively determining the biomarker are applied, the data obtained by said automatically operating means can be processed by, e.g., a computer program in order to facilitate the assessment. Preferably, the means are comprised by a single device in such a case. Said device may accordingly include an analyzing unit for the biomarker and a computer unit for processing the resulting data for the assessment. Alternatively, where means such as test stripes are used for determining the biomarker, the means for comparison may comprise control stripes or tables allocating the determined result data to result data known to be indicative for a medical condition as discussed above. Preferred devices are those which can be applied without the particular knowledge of a specialized clinician, e.g., test stripes or electronic devices which merely require loading with a sample.

Alternatively, the methods for the determination of the at least one biomarker can be implemented into a system comprising several devices which are, preferably, operatively linked to each other. Specifically, the means must be linked in a manner as to allow carrying out the method of the present invention as described in detail above. Therefore, operatively linked, as used herein, preferably, means functionally linked. Depending on the means to be used for the system of the present invention, said means may be functionally linked by connecting each mean with the other by means which allow data transport in between said means, e.g., glass fiber cables, and other cables for high throughput data transport. Nevertheless, wireless data transfer between the means is also envisaged by the present invention, e.g., via LAN (Wireless LAN, WLAN). A preferred system comprises means for determining biomarkers. Means for determining biomarkers as used herein encompass means for separating biomarkers, such as chromatographic devices, and means for metabolite determination, such as mass spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS are used in the system of the present invention as described in detail elsewhere in the specification. Further comprised shall be means for comparing and/or analyzing the results obtained from the means for determination of biomarkers. The means for comparing and/or analyzing the results may comprise at least one databases and an implemented computer program for comparison of the results. Preferred embodiments of the aforementioned systems and devices are also described in detail below.

Furthermore, the present invention relates to a data collection comprising characteristic values of at least one biomarker being indicative for a medical condition or effect as set forth above (i.e. assessing whether gastric bypass was successful, predicting whether gastric bypass will be beneficial, determining whether a supportive therapy accompanying gastric bypass has beneficial effects, diagnosing diabetes, diagnosing body lean mass, diagnosing the energy state or identifying a treatment).

The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for a medical condition or effect as set forth above (e.g. a query search). Thus, if an identical or similar data set can be identified in the data collection, the test data set will be associated with the said medical condition or effect. Consequently, the information obtained from the data collection can be used, e.g., as a reference for the methods of the present invention described above. More preferably, the data collection comprises characteristic values of all metabolites comprised by any one of the groups recited above.

In light of the foregoing, the present invention encompasses a data storage medium comprising the aforementioned data collection.

The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising:
(a) means for comparing characteristic values of the at least one biomarker of a sample operatively linked to
(b) a data storage medium as described above.

The term “system” as used herein relates to different means which are operatively linked to each other. Said means may be implemented in a single device or may be physically separated devices which are operatively linked to each other. The means for comparing characteristic values of biomarkers, preferably, based on an algorithm for comparison as mentioned before. The data storage medium, preferably, comprises the aforementioned data collection or database, wherein each of the stored data sets being indicative for a medical condition or effect referred to above. Thus, the system of the present invention allows identifying whether a test data set is comprised by the data collection stored in the data storage medium. Consequently, the methods of the present invention can be implemented by the system of the present invention.

In a preferred embodiment of the system, means for determining characteristic values of biomarkers of a sample are comprised. The term “means for determining characteristic values of biomarkers” preferably relates to the aforementioned devices for the determination of metabolites such as mass spectrometry devices, NMR devices or devices for carrying out chemical or biological assays for the biomarkers.

Moreover, the present invention relates to a diagnostic means comprising means for the determination of at least one biomarker selected from any one of the groups referred to above.

The term “diagnostic means”, preferably, relates to a diagnostic device, system or biological or chemical assay as specified elsewhere in the description in detail.

The expression “means for the determination of at least one biomarker” refers to devices or agents which are capable of specifically recognizing the biomarker. Suitable devices may be spectrometric devices such as mass spectrometry, NMR devices or devices for carrying out chemical or biological assays for the biomarkers. Suitable agents may be compounds which specifically detect the biomarkers. Detection as used herein may be a two-step process, i.e. the compound may first bind specifically to the biomarker to be detected and subsequently generate a detectable signal, e.g., fluorescent signals, chemiluminescent signals, radioactive signals and the like. For the generation of the detectable signal further compounds may be required which are all comprised by the term “means for determination of the at least one biomarker”. Compounds which specifically bind to the biomarker are described elsewhere in the specification in detail and include, preferably, enzymes, antibodies, ligands, receptors or other biological molecules or chemicals which specifically bind to the biomarkers.

Further, the present invention relates to a diagnostic composition comprising at least one biomarker selected from any one of the groups referred to above.

The at least one biomarker selected from any of the aforementioned groups will serve as a biomarker, i.e. an indicator molecule for a medical condition or effect in the subject as set for the elsewhere herein. Thus, the metabolite molecules itself may serve as diagnostic compositions, preferably, upon visualization or detection by the means referred to in herein. Thus, a diagnostic composition which indicates the presence of a biomarker according to the present invention may also comprise the said biomarker physically, e.g., a complex of an antibody and the metabolite to be detected may serve as the diagnostic composition. Accordingly, the diagnostic composition may further comprise means for detection of the metabolites as specified elsewhere in this description. Alternatively, if detection means such as MS or NMR based techniques are used, the molecular species which serves as an indicator for the risk condition will be the at least one biomarker comprised by the test sample to be investigated. Thus, the at least one biomarker referred to in accordance with the present invention shall serve itself as a diagnostic composition due to its identification as a biomarker.

The biomarkers to be determined in accordance with the methods of the present invention are listed in the following tables. Biomarkers not precisely defined by their name are further characterized in tables 13 and 14.

TABLE 1A
Metabolites changed significantly at 3 months after surgery vs. 0 (pre-surgery)
or 6 months vs. 0 (pre-surgery), in either the diabetic/obese (n = 5) or the
non-diabetic/obese subgroup (n = 9).

| Table 1A | | | | | |
|---|---|---|---|---|---|
| Metabolite | p-value min. | p-value max. | ratio min. | ratio max. | regulation |
| Asparagine | 0.000000 | 0.000000 | 0.478 | 0.478 | down |
| Valine | 0.000000 | 0.000100 | 0.618 | 0.725 | down |
| Kynurenic acid | 0.000000 | 0.000166 | 0.423 | 0.557 | down |
| MetID 0443 | 0.000000 | 0.000000 | 1.499 | 1.499 | up |
| Taurine | 0.000000 | 0.001079 | 0.385 | 0.549 | down |
| Arginine | 0.000000 | 0.000009 | 0.615 | 0.701 | down |
| Sphingomyelin #1 | 0.000000 | 0.000003 | 1.323 | 1.421 | up |
| Tyrosine | 0.000000 | 0.006294 | 0.553 | 0.725 | down |
| Leucine | 0.000000 | 0.000171 | 0.624 | 0.684 | down |
| erythro-C16-Sphingosine | 0.000000 | 0.000000 | 0.689 | 0.699 | down |
| MetID 0009 | 0.000000 | 0.000006 | 1.258 | 1.394 | up |
| MetID 0433 | 0.000000 | 0.000655 | 1.290 | 1.762 | up |
| Ornithine | 0.000000 | 0.000136 | 0.529 | 0.657 | down |
| Biotin | 0.000001 | 0.000001 | 0.603 | 0.603 | down |
| Creatine | 0.000001 | 0.000077 | 0.575 | 0.673 | down |
| Isoleucine | 0.000001 | 0.001527 | 0.665 | 0.746 | down |
| gamma-Linolenic acid | 0.000001 | 0.000084 | 0.387 | 0.509 | down |
| (C18:cis[6,9,12]3) | | | | | |
| Campesterol | 0.000001 | 0.000002 | 0.389 | 0.393 | down |
| Galactose, lipid fraction | 0.000001 | 0.000001 | 1.325 | 1.325 | up |
| MetID 0021 | 0.000002 | 0.000098 | 1.474 | 1.499 | up |
| 1-Octadecenyl-2- | 0.000002 | 0.000002 | 1.341 | 1.341 | up |
| arachidonoylglycero-3- | | | | | |
| phosphocholine (Plasmalogen) | | | | | |
| DAG (C18:1,C18:2) | 0.000002 | 0.000129 | 1.513 | 1.740 | up |
| Citrate | 0.000004 | 0.000491 | 1.290 | 1.448 | up |
| Arachidonic acid (C20:cis- | 0.000004 | 0.000727 | 1.321 | 1.521 | up |
| [5,8,11,14]4) | | | | | |
| Eicosatrienoic acid (C20:3) | 0.000011 | 0.003339 | 0.571 | 0.719 | down |
| Lysine | 0.000011 | 0.000013 | 0.745 | 0.747 | down |
| 3-Hydroxyindole | 0.000015 | 0.000403 | 1.908 | 2.338 | up |
| Threonine | 0.000024 | 0.000180 | 0.665 | 0.707 | down |
| Coenzyme Q9 | 0.000028 | 0.005002 | 0.462 | 0.630 | down |
| Lactate | 0.000028 | 0.001832 | 0.541 | 0.557 | down |
| Canthaxanthin | 0.000029 | 0.000029 | 2.776 | 2.776 | up |
| TAG #2 | 0.000043 | 0.001192 | 1.282 | 1.398 | up |
| 3-Indoxylsulfuric acid | 0.000046 | 0.000576 | 2.034 | 2.422 | up |
| Ceramide (d18:1/C24:0) | 0.000051 | 0.000060 | 0.673 | 0.677 | down |
| Pentadecanol | 0.000057 | 0.000259 | 0.637 | 0.672 | down |
| Nervonic acid (C24:1) | 0.000071 | 0.000520 | 1.918 | 2.266 | up |
| Proline | 0.000081 | 0.000089 | 0.706 | 0.708 | down |
| Xanthine | 0.000082 | 0.000156 | 0.509 | 0.527 | down |
| beta-Aminoisobutyric acid | 0.000101 | 0.001264 | 1.525 | 1.708 | up |
| Indole-3-lactic acid | 0.000124 | 0.002754 | 0.654 | 0.732 | down |
| Phosphatidylcholine #6 | 0.000159 | 0.003394 | 1.260 | 1.525 | up |
| Myristic acid (C14:0) | 0.000169 | 0.001168 | 0.607 | 0.660 | down |
| Cresol sulfate | 0.000169 | 0.000229 | 6.165 | 12.480 | up |
| erythro-Dihydrosphingosine | 0.000202 | 0.008169 | 2.111 | 2.760 | up |
| 3-Hydroxybutyric acid | 0.000208 | 0.000457 | 2.524 | 2.706 | up |
| Tryptophane | 0.000434 | 0.000434 | 0.700 | 0.700 | down |
| beta-Sitosterol | 0.000643 | 0.006344 | 0.248 | 0.298 | down |
| 3-O-Methyl-sphingosine (*1) | 0.000774 | 0.006322 | 1.628 | 2.293 | up |
| 5-O-Methyl-sphingosine (*1) | 0.000838 | 0.005254 | 1.587 | 2.165 | up |
| Lycopene | 0.000918 | 0.000918 | 2.754 | 2.754 | up |
| erythro-Sphingosine (*1) | 0.000938 | 0.007607 | 1.523 | 1.975 | up |
| Alanine | 0.001232 | 0.002316 | 0.532 | 0.669 | down |
| MetID 0389 | 0.001386 | 0.001386 | 1.528 | 1.528 | up |
| threo-Sphingosine (*1) | 0.002155 | 0.009300 | 1.494 | 2.054 | up |
| alpha-Ketoisocaproic acid | 0.002581 | 0.002581 | 1.551 | 1.551 | up |
| MetID 0449 | 0.002737 | 0.002737 | 1.463 | 1.463 | up |
| Phenylalanine | 0.003020 | 0.007410 | 0.682 | 0.726 | down |
| TAG (C55H100O6) (e.g. | 0.003757 | 0.005391 | 1.310 | 1.505 | up |
| C16:0,C18:1,C18:2) | | | | | |
| MetID 1283 | 0.003970 | 0.009616 | 2.319 | 2.795 | up |
| Pseudouridine | 0.004075 | 0.004075 | 1.551 | 1.551 | up |
| Docosahexaenoic acid | 0.005006 | 0.008011 | 2.191 | 3.028 | up |
| (C22:cis[4,7,10,13,16,19]6) | | | | | |
| Cystine | 0.005181 | 0.009187 | 1.251 | 1.275 | up |
| Lysophosphatidylcholine | 0.005562 | 0.009973 | 1.274 | 1.468 | up |
| (C16:0) | | | | | |
| N-Acetyl-neuraminic acid, | 0.005993 | 0.008485 | 1.499 | 2.032 | up |
| lipid fraction | | | | | |
| TAG (containing C16:0/C16:1) | 0.007226 | 0.007226 | 0.680 | 0.680 | down |
| Thyroxine | 0.007684 | 0.007684 | 1.298 | 1.298 | up |

(*1) free and from Sphingolipids

TABLE 1B
Metabolites changed significantly at 3 months after surgery vs. 0 (pre-
surgery) or 6 months vs. 0 (pre-surgery), in all 14 patients.

| Table 1B | | | | | |
|---|---|---|---|---|---|
| Metabolite | p-value min. | p-value max. | ratio min. | ratio max. | regulation |
| Coenzyme Q10 | 0.048357 | | 0.942 | | up |
| Indole-3-acetic acid | 0.013732 | 0.032261 | 1.333 | 1.400 | up |
| Palmitoleic acid (C16:cis[9]1) | 0.032863 | 0.032863 | 0.813 | 0.820 | down |
| Phosphatidylcholine (C16:0, | 0.001966 | 0.039628 | 1.013 | 1.021 | up |
| C20:4) | | | | | |
| Phosphatidylcholine (C16:1, | 0.001676 | 0.008306 | 0.802 | 0.835 | down |
| C18:2) | | | | | |
| Phosphatidylcholine (C18:0, | 0.000058 | 0.000727 | 0.893 | 0.914 | down |
| C18:1) | | | | | |
| Phosphatidylcholine #8 | 0.002621 | 0.021649 | 1.034 | 1.046 | up |
| Serine | 0.000896 | 0.001613 | 0.874 | 0.882 | down |
| Stearic acid (C18:0) | 0.000113 | 0.000237 | 0.810 | 0.820 | down |
| Threonic acid | 0.016658 | | 1.253 | | up |
| beta-Carotene | 0.000105 | | 3.144 | | up |
| Elaidic acid | 0.012081 | 0.015506 | 1.524 | 1.829 | up |
| Glycine | 0.028855 | 0.037799 | 1.143 | 1.152 | up |
| Phosphatidylcholine (C16:0, | 0.004806 | 0.049116 | 1.256 | 1.257 | up |
| C16:0) | | | | | |
| Phosphatidylcholine (C18:0, | 0.000006 | 0.000019 | 1.019 | 1.020 | up |
| C18:2) | | | | | |
| Phosphatidylcholine (C18:0, | 0.000008 | 0.000014 | 1.186 | 1.194 | up |
| C22:6) | | | | | |
| Phosphatidylcholine (C18:2, | 4.033E−09 | 5.011E−08 | 1.146 | 1.169 | up |
| C20:4) | | | | | |
| Phosphatidylcholine #3 | 0.000001 | 0.000305 | 1.135 | 1.222 | up |

TABLE 2
Metabolites changed at 3 months after surgery vs. pre-surgery,
in the diabetic/obese subgroup (n = 5).

| Table 2 | | | |
|---|---|---|---|
| Metabolite | p-value | ratio | regulation |
| Asparagine | 0.000000 | 0.478 | down |
| erythro-C16-Sphingosine | 0.000000 | 0.689 | down |
| Biotin | 0.000001 | 0.603 | down |
| Creatine | 0.000001 | 0.575 | down |
| Campesterol | 0.000001 | 0.389 | down |
| MetID 0021 | 0.000002 | 1.474 | up |
| Sphingomyelin #1 | 0.000003 | 1.323 | up |
| Citrate | 0.000004 | 1.448 | up |
| Lysine | 0.000013 | 0.747 | down |
| 3-Hydroxyindole | 0.000015 | 2.338 | up |
| MetID 0433 | 0.000017 | 1.597 | up |
| Threonine | 0.000024 | 0.665 | down |
| Lactate | 0.000028 | 0.541 | down |
| 3-Indoxylsulfuric acid | 0.000046 | 2.422 | up |
| Ceramide (d18:1/C24:0) | 0.000051 | 0.673 | down |
| Pentadecanol | 0.000057 | 0.637 | down |
| Nervonic acid (C24:1) | 0.000071 | 1.918 | up |
| Valine | 0.000100 | 0.725 | down |
| Indole-3-lactic acid | 0.000124 | 0.654 | down |
| DAG (C18:1,C18:2) | 0.000129 | 1.513 | up |
| Myristic acid (C14:0) | 0.000169 | 0.607 | down |
| Cresol sulfate | 0.000169 | 6.165 | up |
| Leucine | 0.000171 | 0.684 | down |
| 3-Hydroxybutyric acid | 0.000208 | 2.706 | up |
| Tryptophane | 0.000434 | 0.700 | down |
| beta-Sitosterol | 0.000643 | 0.298 | down |
| Arachidonic acid | 0.000727 | 1.321 | up |
| (C20:cis-[5,8,11,14]4) | | | |
| 3-O-Methyl-sphingosine | 0.000774 | 1.848 | up |
| (*1) | | | |
| 5-O-Methyl-sphingosine | 0.000838 | 1.771 | up |
| (*1) | | | |
| erythro-Sphingosine (*1) | 0.000938 | 1.676 | up |

(*1) free and from Sphingolipids

TABLE 3
Metabolites correlating significantly with insulin sensitivity (determined
by QUICKI, Yokoyama H et al, Diabetes Care, 2003) at all three time
points (pre-surgery, 3 and 6 months post-surgery). A negative correlation
with insulin sensitivity indicates that up-regulated (increased) metabolite
amounts (in comparison to control groups) are associated with diabetes
or diabetes risk. A positive correlation with insulin sensitivity indicates
that down-regulated metabolite amounts (in comparison to control groups)
are associated with diabetes or diabetes risk. A change towards normal in
metabolite levels after surgery indicates successful gastric bypass therapy.

| Table 3 | | | |
|---|---|---|---|
| Metabolite | p-value | R² | correlation |
| TAG (C55H10006) (e.g. | 0.000029 | 0.36 | positive |
| C16:0,C18:1,C18:2) | | | |
| Ascorbic acid | 0.000002 | 0.45 | negative |
| Glucose | 0.000008 | 0.40 | negative |
| Valine | 0.000008 | 0.40 | negative |
| MetID 0060 | 0.000026 | 0.37 | positive |
| threo-Sphingosine (*1) | 0.000035 | 0.36 | positive |
| Nervonic acid (C24:1) | 0.000047 | 0.35 | positive |
| Linoleic acid | 0.000049 | 0.35 | positive |
| (C18:cis[9,12]2) | | | |
| erythro-Sphingosine (*1) | 0.000054 | 0.35 | positive |
| 3-O-Methyl-sphingosine | 0.000055 | 0.34 | positive |
| (*1) | | | |
| Glucose-1-phosphate | 0.000076 | 0.33 | negative |
| Sorbitol | 0.000395 | 0.33 | negative |
| 5-O-Methyl-sphingosine | 0.000093 | 0.33 | positive |
| (*1) | | | |
| Galactose, lipid fraction | 0.000108 | 0.32 | positive |
| N-Acetyl-neuraminic acid, | 0.000108 | 0.32 | positive |
| lipid fraction | | | |
| TAG (containing | 0.000165 | 0.31 | positive |
| C18:2,C18:2) | | | |
| Sphingomyelin #1 | 0.000168 | 0.31 | positive |
| Phytosphingosine | 0.000171 | 0.31 | positive |
| MetID 0443 | 0.000208 | 0.30 | positive |

(*1) free and from Sphingolipids

TABLE 4
Metabolites changed significantly at 3 months after surgery vs. 0 (pre-surgery)
or 6 months vs. pre-surgery, in the nondiabetic/obese subgroup (n = 9).

| Table 4 | | | | | |
|---|---|---|---|---|---|
| Metabolite | p-value min. | p-value max. | ratio min. | ratio max. | regulation |
| Kynurenic acid | 0.000000 | 0.000000 | 0.423 | 0.497 | down |
| Valine | 0.000000 | 0.000000 | 0.618 | 0.634 | down |
| Arginine | 0.000000 | 0.000009 | 0.615 | 0.701 | down |
| Sphingomyelin #1 | 0.000000 | 0.000003 | 1.323 | 1.421 | up |
| Tyrosine | 0.000000 | 0.000000 | 0.553 | 0.563 | down |
| Leucine | 0.000000 | 0.000000 | 0.624 | 0.631 | down |
| erythro-C16-Sphingosine | 0.000000 | 0.000000 | 0.689 | 0.699 | down |
| MetID 0433 | 0.000000 | 0.000655 | 1.290 | 1.593 | up |
| Ornithine | 0.000000 | 0.000136 | 0.529 | 0.657 | down |
| Creatine | 0.000001 | 0.000077 | 0.575 | 0.673 | down |
| Isoleucine | 0.000001 | 0.000005 | 0.665 | 0.698 | down |
| gamma-Linolenic acid | 0.000001 | 0.000084 | 0.387 | 0.509 | down |
| (C18:cis[6,9,12]3) | | | | | |
| Campesterol | 0.000001 | 0.000002 | 0.389 | 0.393 | down |
| MetID 0021 | 0.000002 | 0.000098 | 1.474 | 1.499 | up |
| DAG (C18:1,C18:2) | 0.000002 | 0.000129 | 1.513 | 1.740 | up |
| Citrate | 0.000004 | 0.000491 | 1.290 | 1.448 | up |
| Arachidonic acid (C20:cis- | 0.000004 | 0.000727 | 1.321 | 1.521 | up |
| [5,8,11,14]4) | | | | | |
| Eicosatrienoic acid (C20:3) | 0.000011 | 0.003339 | 0.571 | 0.719 | down |
| Lysine | 0.000011 | 0.000013 | 0.745 | 0.747 | down |
| 3-Hydroxyindole | 0.000015 | 0.000403 | 1.908 | 2.338 | up |
| Threonine | 0.000024 | 0.000180 | 0.665 | 0.707 | down |
| Coenzyme Q9 | 0.000028 | 0.005002 | 0.462 | 0.630 | down |
| Lactate | 0.000028 | 0.001832 | 0.541 | 0.557 | down |
| TAG #2 | 0.000043 | 0.001192 | 1.282 | 1.398 | up |
| 3-Indoxylsulfuric acid | 0.000046 | 0.000576 | 2.034 | 2.422 | up |
| Ceramide (d18:1/C24:0) | 0.000051 | 0.000060 | 0.673 | 0.677 | down |
| Pentadecanol | 0.000057 | 0.000259 | 0.637 | 0.672 | down |
| Nervonic acid (C24:1) | 0.000071 | 0.000520 | 1.918 | 2.266 | up |
| Proline | 0.000081 | 0.000089 | 0.706 | 0.708 | down |
| Xanthine | 0.000082 | 0.000156 | 0.509 | 0.527 | down |
| beta-Aminoisobutyric acid | 0.000101 | 0.001264 | 1.525 | 1.708 | up |
| Indole-3-lactic acid | 0.000124 | 0.002754 | 0.654 | 0.732 | down |
| Myristic acid (C14:0) | 0.000169 | 0.001168 | 0.607 | 0.660 | down |
| Cresol sulfate | 0.000169 | 0.000229 | 6.165 | 12.480 | up |
| 3-Hydroxybutyric acid | 0.000208 | 0.000457 | 2.524 | 2.706 | up |
| beta-Sitosterol | 0.000643 | 0.006344 | 0.248 | 0.298 | down |

TABLE 5
Table 5: Metabolites correlating significantly with body fat
mass (in % of total body mass) at all time points (pre-surgery,
3 and 6 months post-surgery). A positive correlation with %
body fat mass indicates that up-regulated metabolite amounts
(in comparison to control groups) are associated with obesity
or obesity risk. A negative correlation with % body fat mass
indicates that down-regulated (decreased) metabolite amounts
(in comparison to control groups) are associated with obesity
or obesity risk. A change towards normal in metabolite levels
after surgery indicates successful gastric bypass therapy.

| Metabolite | p-value | R² | Correlation |
|---|---|---|---|
| alpha-Ketoisocaproic acid | 0.000015 | 0.38 | negative |
| 3-Methoxy-tyrosine | 0.000073 | 0.33 | positive |
| Glycerol, polar fraction | 0.000076 | 0.33 | positive |
| Phosphatidylcholine (C18:1/C18:2) | 0.000399 | 0.27 | negative |
| 3-Indoxylsulfuric acid | 0.000984 | 0.24 | negative |
| Coenzyme Q9 | 0.001675 | 0.22 | positive |
| MetID 0389 | 0.002103 | 0.21 | negative |
| MetID 0449 | 0.002627 | 0.20 | negative |
| Cresol sulfate | 0.003361 | 0.20 | negative |
| Lysophosphatidylcholine (C16:0) | 0.003880 | 0.19 | negative |
| Glutamate | 0.005605 | 0.18 | positive |
| Serotonine | 0.006217 | 0.17 | negative |
| Tricosanoic acid (C23:0) | 0.009303 | 0.16 | negative |
| 4-Hydroxy-3-methoxy-mandelic acid | 0.010250 | 0.15 | positive |

TABLE 6
Table 6: Metabolite levels at the pre-surgery time point
correlating with the change in % body fat mass, comparing
12 months after surgery with pre-surgery values. A positive
correlation with the change in % body fat mass indicates that
up-regulated metabolite amounts (in comparison to control
groups) at the pre-surgery time point predict successful
gastric bypass therapy. A negative correlation with the change
in % body fat mass indicates that down-regulated (decreased)
metabolite amounts (in comparison to control groups) at the
pre-surgery time point predict successful gastric
bypass therapy.

| Metabolite | p-value | R² | Correlation |
|---|---|---|---|
| beta-Aminoisobutyric acid | 0.01537 | 0.43 | positive |
| Phosphatidylcholine plasmalogenes | 0.01993 | 0.40 | positive |
| Dihydrocholesterol | 0.02018 | 0.40 | positive |
| Phosphatidylcholine #10 | 0.02713 | 0.37 | positive |
| MetID 0430 | 0.02919 | 0.36 | positive |
| Valine | 0.03176 | 0.35 | negative |
| Hexadecanol | 0.03204 | 0.35 | positive |
| Cholesterolester | 0.03319 | 0.35 | negative |
| Phosphatidylcholine #6 | 0.03521 | 0.34 | positive |
| Phosphatidylcholine #9 | 0.03732 | 0.34 | positive |
| Cysteine | 0.03976 | 0.33 | negative |
| Lysophosphatidylethanolamine | 0.04081 | 0.33 | positive |
| Alanine | 0.04607 | 0.31 | negative |
| Sphingomyelin #2 | 0.04645 | 0.31 | positive |
| Lactate | 0.04878 | 0.31 | negative |
| Tyrosine | 0.05049 | 0.30 | negative |
| Tryptophane | 0.05073 | 0.30 | negative |

TABLE 7
Table 7: Metabolite levels at the pre-surgery time point
correlating with the change in insulin sensitivity (determined
by QUICKI), comparing 12 months after surgery with pre-surgery
values. A positive correlation with the change in insulin
sensitivity indicates that up-regulated (increased) metabolite
amounts (in comparison to control groups) at the pre-surgery
time point predict successful gastric bypass therapy. A
negative correlation with the change in insulin sensitivity
indicates that down-regulated (decreased) metabolite amounts
(in comparison to control groups) at the pre-surgery time
point predict successful gastric bypass therapy.

| Metabolite | p-value | R² | Correlation |
|---|---|---|---|
| Arachidonic acid (C20:cis- | 0.003724 | 0.63 | positive |
| [5,8,11,14]4) | | | |
| Heptadecanoic acid (C17:0) | 0.007899 | 0.56 | positive |
| Cryptoxanthin | 0.007912 | 0.56 | positive |
| Cholesterol | 0.010220 | 0.63 | positive |
| beta-Aminoisobutyric acid | 0.019190 | 0.47 | positive |
| Phosphatidylcholine | 0.021220 | 0.46 | positive |
| (C18:0/C22:6) | | | |
| Isoleucine | 0.022540 | 0.46 | positive |
| MetID 0052 | 0.025850 | 0.44 | positive |
| Leucine | 0.027170 | 0.44 | positive |
| Phosphatidylcholine | 0.027880 | 0.43 | positive |
| (C18:2/C20:4) | | | |
| myo-Inositol-phosphates, lipid | 0.029420 | 0.43 | positive |
| fraction | | | |
| Docosahexaenoic acid | 0.029820 | 0.42 | positive |
| (C22:cis[4,7,10,13,16,19]6) | | | |
| Sorbitol | 0.031940 | 0.42 | positive |
| Phosphatidylcholine #8 | 0.032160 | 0.42 | positive |
| beta-Sitosterol | 0.034070 | 0.41 | positive |
| beta-Carotene | 0.037780 | 0.40 | positive |
| Urea | 0.039480 | 0.39 | positive |
| Lignoceric acid (C24:0) | 0.040150 | 0.39 | positive |
| Androstenedione | 0.051060 | 0.36 | negative |
| Testosterone-17-sulfate | 0.058860 | 0.34 | negative |
| Histidine | 0.059450 | 0.34 | positive |
| Phosphatidylcholine #9 | 0.059770 | 0.34 | positive |
| Creatine | 0.065010 | 0.33 | positive |
| Testosterone | 0.067760 | 0.32 | negative |
| Lysine | 0.072630 | 0.31 | positive |
| Behenic acid (C22:0) | 0.074950 | 0.31 | positive |
| Cortisol | 0.075780 | 0.31 | negative |
| Tricosanoic acid (C23:0) | 0.077890 | 0.31 | positive |
| Citrulline | 0.077950 | 0.31 | positive |

TABLE 8
Table 8: Metabolites reduced at 3 months post-surgery
compared to pre-surgery, filtered for exogenous,
preferably essential exogenous nutrients.

| Metabolite | p-value | ratio | regulation |
|---|---|---|---|
| Asparagine | 0.000000 | 0.478 | down |
| Valine | 0.000000 | 0.618 | down |
| Taurine | 0.000000 | 0.385 | down |
| Leucine | 0.000000 | 0.624 | down |
| Tyrosine | 0.000000 | 0.563 | down |
| Biotin | 0.000001 | 0.603 | down |
| gamma-Linolenic acid | 0.000001 | 0.387 | down |
| (C18:cis[6,9,12]3) | | | |
| Campesterol | 0.000001 | 0.389 | down |
| Isoleucine | 0.000005 | 0.698 | down |
| Lysine | 0.000013 | 0.747 | down |
| Threonine | 0.000024 | 0.665 | down |
| Coenzyme Q9 | 0.000028 | 0.462 | down |
| Myristic acid (C14:0) | 0.000169 | 0.607 | down |
| Tryptophane | 0.000434 | 0.700 | down |
| beta-Sitosterol | 0.000643 | 0.298 | down |
| Phenylalanine | 0.003020 | 0.726 | down |
| Salicylic acid | 0.034856 | 0.660 | down |
| Eicosapentaenoic acid | 0.035061 | 0.725 | down |
| (C20:cis[5,8,11,14,17]5) | | | |

TABLE 9
Table 9: Metabolites differing between diabetic/obese and
nondiabetic/obese at the pre-surgery time point t0 or post-
surgery time points t3 or t6.

| | p-value | p-value | ratio | ratio | |
|---|---|---|---|---|---|
| Metabolite | MIN | MAX | MIN | MAX | regulation |
| 1,5-Anhydrosorbitol | 0.000042 | 0.0032 | 0.208 | 0.404 | down |
| 11-Deoxycortisol | 0.0032 | | 3.567 | | up |
| 3-O-Methylsphing- | 0.0176 | | 0.722 | | down |
| osine (*1) | | | | | |
| 5-Hydroxy-3-indoleacetic | 0.0116 | | 1.553 | | up |
| acid | | | | | |
| 5-O-Methylsphing- | 0.0249 | | 0.754 | | down |
| osine (*1) | | | | | |
| Androstenedione | 0.0062 | | 0.298 | | down |
| Arginine | 0.0469 | | 0.741 | | down |
| Ascorbic acid | 0.002 | 0.002 | 1.318 | 1.318 | up |
| Asparagine | 0.0046 | 0.0046 | 1.379 | 1.379 | up |
| Citrulline | 0.0241 | 0.0439 | 1.394 | 1.463 | up |
| Cresol sulfate | 0.0133 | 0.0133 | 1.962 | 1.962 | up |
| Cysteine | 0.0091 | 0.0091 | 1.353 | 1.353 | up |
| D-Threitol | 0.0241 | 0.0366 | 1.692 | 1.781 | up |
| erythro-Sphingosine (*1) | 0.0243 | | 0.769 | | down |
| Fructose-6-phosphate | 0.0296 | 0.0296 | 1.461 | 1.461 | up |
| gamma-Linolenic acid | 0.03 | | 1.867 | | up |
| (C18:cis[6,9,12]3) | | | | | |
| Glucose | 0.0376 | 0.0376 | 1.235 | 1.235 | up |
| Glucose-1-phosphate | 0.0167 | 0.0167 | 1.438 | 1.438 | up |
| Hypoxanthine | 0.0041 | | 2.231 | | up |
| Isoleucine | 0.0209 | | 1.308 | | up |
| Lactate | 0.014 | 0.014 | 1.201 | 1.201 | up |
| Leucine | 0.003 | | 1.478 | | up |
| Linoleic acid | 0.007 | | 0.628 | | down |
| (C18:cis[9,12]2) | | | | | |
| Lycopene | 0.0043 | 0.0043 | 0.444 | 0.444 | down |
| Lysophosphatidylcholine | 0.0059 | 0.0059 | 0.779 | 0.779 | down |
| (C18:2) | | | | | |
| Mannose | 0.0468 | 0.0468 | 1.196 | 1.196 | up |
| myo-Inositol-2-phosphate | 0.0033 | 0.0195 | 1.271 | 1.385 | up |
| N-Acetylneuraminic | 0.0256 | | 0.732 | | down |
| acid, lipid fraction | | | | | |
| Nervonic acid (C24:1) | 0.0265 | 0.0265 | 0.781 | 0.781 | down |
| Normetanephrine | 0.004 | | 0.21 | | down |
| Ornithine | 0.0237 | 0.0409 | 1.407 | 1.47 | up |
| Pantothenic acid | 0.0176 | 0.0176 | 1.668 | 1.668 | up |
| Phenylalanine | 0.0112 | | 1.274 | | up |
| Phosphatidylcholine #3 | 0.0083 | | 1.285 | | up |
| Phosphatidylcholine | 0.0498 | | 0.874 | | down |
| (C16:0, C16:0) | | | | | |
| Phosphatidylcholine | 0.0091 | 0.0091 | 1.025 | 1.025 | up |
| (C16:0, C18:2) | | | | | |
| Phosphatidylcholine | 0.0064 | 0.0223 | 1.096 | 1.122 | up |
| (C18:0, C18:1) | | | | | |
| Phosphatidylcholine | 0.0327 | | 1.027 | | up |
| (C18:1, C18:2) | | | | | |
| Phosphatidylcholine | 0.0146 | | 1.119 | | up |
| (C18:2, C20:4) | | | | | |
| Phytosphingosine | 0.0302 | | 0.665 | | down |
| Proline | 0.0061 | | 1.452 | | up |
| Ribose | 0.0148 | | 2.06 | | up |
| Sphingomyelin #2 | 0.0484 | | 0.945 | | down |
| Sucrose | 0.039 | | 2.738 | | up |
| Taurine | 0.0181 | 0.0309 | 1.444 | 1.509 | up |
| Testosterone | 0.0024 | | 0.421 | | down |
| Testosterone-17-sulfate | 0.0299 | 0.0299 | 0.439 | 0.439 | down |
| threo-Sphingosine (*1) | 0.0232 | | 0.75 | | down |
| Valine | 0.0004 | 0.0115 | 1.263 | 1.465 | up |
| Xanthine | 0.0082 | 0.0264 | 1.533 | 1.706 | up |

(*1) free and from Sphingolipids

TABLE 10
Table 10: Metabolites correlating significantly with insulin
sensitivity (determined by QUICKI) at all three time points
(pre-surgery, 3 and 6 months post-surgery). A negative
correlation with insulin sensitivity indicates that up-
regulated (increased) metabolite amounts (in comparison to
control groups) are associated with diabetes or diabetes risk.
A positive correlation with insulin sensitivity indicates
that down-regulated (decreased) metabolite amounts (in
comparison to control groups) are associated with diabetes
or diabetes risk.

| Metabolite | p-value | R² | |
|---|---|---|---|
| TAG (C55H100O6) (e.g. | 0.000029 | 0.36 | positive |
| C16:0, C18:1, C18:2) | | | |
| MetID 0060 | 0.000026 | 0.37 | positive |
| threo-Sphingosine (*1) | 0.000035 | 0.36 | positive |
| erythro-Sphingosine (*1) | 0.000054 | 0.35 | positive |
| 3-O-Methyl-sphingosine (*1) | 0.000055 | 0.34 | positive |
| Glucose-1-phosphate | 0.000076 | 0.33 | negative |
| Sorbitol | 0.000395 | 0.33 | negative |
| 5-O-Methyl-sphingosine (*1) | 0.000093 | 0.33 | positive |
| Galactose, lipid fraction | 0.000108 | 0.32 | positive |
| N-Acetyl-neuraminic acid, lipid | 0.000108 | 0.32 | positive |
| fraction | | | |
| TAG (containing C18:2, C18:2) | 0.000165 | 0.31 | positive |
| Sphingomyelin #1 | 0.000168 | 0.31 | positive |
| Phytosphingosine | 0.000171 | 0.31 | positive |
| MetID 0443 | 0.000208 | 0.30 | positive |

(*1) free and from Sphingolipids

TABLE 11
Table 11: Metabolites correlating with resting energy
expenditure at all 3 time points (pre-surgery, 3 and 6 months
post-surgery). A negative correlation with resting energy
expenditure indicates that up-regulated (increased)
metabolite amounts (in comparison to control groups) are
associated with negative energy state. A positive correlation
with resting energy expenditure indicates that down-regulated
(decreased) metabolite amounts (in comparison to control
groups) are associated with negative energy state.

| Metabolite | p-value | R² | Correlation |
|---|---|---|---|
| Lysophosphatidylcholine | 0.000001 | 0.46 | negative |
| (C16:0) | | | |
| MetID 0433 | 0.000014 | 0.38 | negative |
| Kynurenic acid | 0.000019 | 0.37 | negative |
| Pseudouridine | 0.000027 | 0.36 | positive |
| MetID 0389 | 0.000029 | 0.36 | negative |
| MetID 0449 | 0.00003 | 0.36 | negative |
| MetID 0060 | 0.000072 | 0.33 | negative |
| MetID 0021 | 0.000101 | 0.32 | negative |
| Phosphatidylcholine | 0.00012 | 0.31 | negative |
| (C18:0, C18:2) | | | |
| DAG (C18:1, C18:2) | 0.00016 | 0.3 | negative |
| Alanine | 0.000291 | 0.28 | positive |
| Dodecanol | 0.000384 | 0.27 | negative |
| Nervonic acid (C24:1) | 0.000427 | 0.27 | negative |
| Glutamine | 0.000659 | 0.25 | negative |
| Sorbitol | 0.00088 | 0.29 | positive |
| MetID 0132 | 0.000929 | 0.24 | negative |
| Cresol sulfate | 0.001009 | 0.24 | negative |
| Lactate | 0.001084 | 0.24 | positive |
| myo-Inositol-phosphates, | 0.001105 | 0.24 | negative |
| lipid fraction | | | |
| Phosphatidylcholine | 0.001139 | 0.24 | negative |
| (C16:0/C16:0) | | | |
| Phosphatidylcholine | 0.001768 | 0.22 | negative |
| (C18:2/C20:4) | | | |
| Docosahexaenoic acid | 0.00186 | 0.22 | negative |
| (C22:cis[4,7,10,13,16,19]6) | | | |
| TAG (C55H100O6) (e.g. | 0.002013 | 0.21 | negative |
| C16:0, C18:1, C18:2) | | | |
| MetID 0443 | 0.002091 | 0.21 | negative |
| Elaidic acid | 0.002197 | 0.21 | negative |
| Glycine | 0.002252 | 0.21 | negative |
| Palmitic acid (C16:0) | 0.002676 | 0.2 | negative |
| Citrate | 0.002697 | 0.2 | negative |

TABLE 12
Table 12: Metabolites correlating with % body lean mass at
all 3 time points (pre-surgery, 3 and 6 months post-surgery).
A positive correlation with % body lean mass indicates that
up-regulated (increased) metabolite amounts (in comparison to
control groups) are associated with high % body lean mass. A
negative correlation with % body lean mass indicates that
down-regulated (decreased) metabolite amounts (in comparison
to control groups) are associated with high % body lean mass.

| Metabolite | p-value | R² | Correlation |
|---|---|---|---|
| alpha-Ketoisocaproic acid | 0.000016 | 0.38 | positive |
| 3-Methoxy-tyrosine | 0.000081 | 0.33 | negative |
| Glycerol, polar fraction | 0.000088 | 0.32 | negative |
| Phosphatidylcholine (C18:1/C18:2) | 0.000371 | 0.27 | positive |
| 3-Indoxylsulfuric acid | 0.001061 | 0.24 | positive |
| Coenzyme Q9 | 0.002195 | 0.21 | negative |
| MetID 0389 | 0.002365 | 0.21 | positive |
| MetID 0449 | 0.002989 | 0.20 | positive |
| Cresol sulfate | 0.004393 | 0.19 | positive |
| Lysophosphatidylcholine (C16:0) | 0.004985 | 0.18 | positive |
| Serotonine | 0.006188 | 0.17 | positive |
| Glutamate | 0.006869 | 0.17 | negative |
| Tricosanoic acid (C23:0) | 0.007988 | 0.16 | positive |
| Uric acid | 0.010430 | 0.15 | negative |
| 4-Hydroxy-3-methoxy-mandelic acid | 0.011260 | 0.15 | negative |

TABLE 13
Chemical/physical properties of “Unkowns”. The biomarkers defined by a
MetID in the previous tables 1 to 12 are characterized by chemical and physical properties.

| MetID | m/z ratio | Fragmentation pattern GC |
|---|---|---|
| MetID 1283 | 71 | metID 1283 which is present in human serum and if detected |
| | | with GC/MS analysis with application of an electron impact mass |
| | | spectrometry at 70 eV and after acidic methanolysis and derivatisation |
| | | with 2% O-methylhydroxylamine-hydrochlorid in pyridine |
| | | and subsequently with N-methyl-N-trimethylsilyltrifluoracetamid |
| | | has the following characteristic nominal masses (relative ratios): |
| | | 71 (100 +/− 20%), 72 (82 +/− 20%), 58 (41 +/− 20%), |
| | | 73 (16 +/− 20%) |
| MetID 0389 | 154 | metID 0389 which is present in human serum and if detected |
| | | with GC/MS analysis with application of an electron impact mass |
| | | spectrometry at 70 eV and after acidic methanolysis and derivatisation |
| | | with 2% O-methylhydroxylamine-hydrochlorid in pyridine |
| | | and subsequently with N-methyl-N-trimethylsilyltrifluoracetamid |
| | | has the following characteristic nominal masses (relative ratios): |
| | | 154 (100 +/− 20%), 75 (50 +/− 20%), 155 (12 +/− 20%) |
| MetID 0449 | 156 | metID 0449 which is present in human serum and if detected |
| | | with GC/MS analysis with application of an electron impact mass |
| | | spectrometry at 70 eV and after acidic methanolysis and derivatisation |
| | | with 2% O-methylhydroxylamine-hydrochlorid in pyridine |
| | | and subsequently with N-methyl-N-trimethylsilyltrifluoracetamid |
| | | has the following characteristic nominal masses (relative ratios): |
| | | 156 (100 +/− 20%), 73 (63 +/− 20%), 157 (36 +/− 20%), |
| | | 45 (11 +/− 20%), 75 (11 +/− 20%) |
| MetID 0151 | 412.6 | |
| MetID 0009 | 729.8 | |
| MetID 0021 | 426.4 | |
| MetID 0022 | 801.8 | |
| MetID 0052 | 811.6 | |
| MetID 0060 | 991.8 | |
| MetID 0430 | 853.6 | |
| MetID 0433 | 879.6 | |
| MetID 0435 | 369.2 | |
| MetID 0443 | 904 | |

TABLE 14
Chemical/physical properties of selected analytes. These biomarkers are
characterized herein by chemical and physical properties.

| Name | m/z ratio | Fragmentation pattern (GCMS) and description |
|---|---|---|
| 1-Octadecenyl-2- | 795 | 1-Octadecenyl-2-arachidonoylglycero-3-phosphocholine |
| arachidonoylglycero- | | (Plasmalogen) represents the sum parameter of glycerophosphorylcholine |
| 3- | | plasmalogens. The mass-to-charge |
| phosphocholine | | ratio (m/z) of the ionised species is 795.0 Da |
| (Plasmalogen) | | (+/−0.5 Da). |
| 3-Indoxylsulfuric | 212.2 | |
| acid | | |
| Ceramide | 650.8 | Ceramide (d18:1/C24:0) represents the sum parameter |
| (d18:1/C24:0) | | of ceramides containing the combination of a d18:1 long- |
| | | chain base unit and a C24:0 fatty acid unit. The mass-to- |
| | | charge ratio (m/z) of the ionised species is 650.8 Da |
| | | (+/−0.5 Da). |
| Cholesterolester | 369.2 | Cholesterolester represents the sum parameter of cholesterol |
| | | esters. The mass-to-charge ratio (m/z) of the |
| | | ionised species is 369.2 Da (+/−0.5 Da). |
| Cresol sulfate | 186.6 | Cresol sulfate represents the sum parameter of ortho-/ |
| | | meta- and para-Cresol sulfates |
| DAG (C18:1, C18:2) | 641.6 | DAG (C18:1, C18:2) represents the sum parameter of |
| | | diacylglycerols containing the combination of a C18:1 |
| | | fatty acid unit and a C18:2 fatty acid unit. The mass-to- |
| | | charge ratio (m/z) of the ionised species is 641.6 Da |
| | | (+/−0.5 Da). |
| Lysophosphatidyl- | 510.4 | Lysophosphatidylethanolamine represents the sum parameter |
| ethanolamine | | of glycerolysophosphorylethanolamine. The |
| | | mass-to-charge ratio (m/z) of the ionised species is |
| | | 510.4 Da (+/−0.5 Da). |
| 3-O-Methyl- | 204 | 3-O-Methyl-sphingosine which is present in human serum |
| sphingosine | | and if detected with GC/MS analysis with application |
| | | of an electron impact mass spectrometry at 70 eV and |
| | | after acidic methanolysis and derivatisation with 2% O- |
| | | methylhydroxylamine-hydrochlorid in pyridine and subsequently |
| | | with N-methyl-N-trimethylsilyltrifluoracetamid |
| | | has the following characteristic nominal masses (relative |
| | | ratios): 204 (100 +/− 20%), 73 (18 +/− 20%), |
| | | 205 (16 +/− 20%), 206 (7 +/− 20%), 354 (4 +/− 20%), |
| | | 442 (1 +/− 20%) |
| 5-O-Methyl- | 250 | 5-O-Methyl-sphingosine which is present in human serum |
| sphingosine | | and if detected with GC/MS analysis with application |
| | | of an electron impact mass spectrometry at 70 eV and |
| | | after acidic methanolysis and derivatisation with 2% O- |
| | | methylhydroxylamine-hydrochlorid in pyridine and subsequently |
| | | with N-methyl-N-trimethylsilyltrifluoracetamid |
| | | has the following characteristic nominal masses (relative |
| | | ratios): 250 (100 +/− 20%), 73 (34 +/− 20%), |
| | | 251 (19 +/− 20%), 354 (14 +/− 20%), 355 (4 +/− 20%), |
| | | 442 (1 +/− 20%) |
| Phosphatidylcholine | 772.6 | Phosphatidylcholine #10 represents the sum parameter |
| #10 | | of glycerophosphorylcholine plasmalogens. The mass- |
| | | to-charge ratio (m/z) of the ionised species is 772.6 Da |
| | | (+/−0.5 Da). |
| Phosphatidylcholine | 808.4 | Phosphatidylcholine #3 represents the sum parameter of |
| #3 | | glycerophosphorylcholines. The total number of carbon |
| | | atoms and the total number of double bonds of the two |
| | | fatty acid moieties together is 38 and 5, respectively. The |
| | | mass-to-charge ratio (m/z) of the ionised species is |
| | | 808.4 Da (+/−0.5 Da). |
| Phosphatidylcholine | 767 | Phosphatidylcholine #6 represents the sum parameter of |
| #6 | | glycerophosphorylcholine plasmalogens. The mass-to- |
| | | charge ratio (m/z) of the ionised species is 767.0 Da |
| | | (+/−0.5 Da). |
| Phosphatidylcholine | 810.8 | Phosphatidylcholine #8 represents the sum parameter of |
| #8 | | glycerophosphorylcholines containing the combination of |
| | | a C18:0 fatty acid unit and a C20:4 fatty acid unit. The |
| | | mass-to-charge ratio (m/z) of the ionised species is |
| | | 810.8 Da (+/−0.5 Da). |
| Phosphatidylcholine | 796.8 | Phosphatidylcholine #9 represents the sum parameter of |
| #9 | | glycerophosphorylcholines. The mass-to-charge ratio |
| | | (m/z) of the ionised species is 796.8 Da (+/−0.5 Da). |
| Phosphatidylcholine | 734.8 | Phosphatidylcholine (C16:0/C16:0) represents the sum |
| (C16:0/C16:0) | | parameter of glycerophosphorylcholines containing either |
| | | the combination of of two C16:0 fatty acid units. |
| | | The mass-to-charge ratio (m/z) of the ionised species is |
| | | 734.8 Da (+/−0.5 Da). |
| Phosphatidylcholine | 784.6 | Phosphatidylcholine (C18:1/C18:2) represents the sum |
| (C18:1/C18:2) | | parameter of glycerophosphorylcholines containing the |
| | | combination of a C18:1 fatty acid unit and a C18:2 fatty |
| | | acid unit. The mass-to-charge ratio (m/z) of the ionised |
| | | species is 784.6 Da (+/−0.5 Da). |
| Phosphatidylcholine | 806.8 | Phosphatidylcholine (C18:2/C20:4) represents the sum |
| (C18:2/C20:4) | | parameter of glycerophosphorylcholines containing either |
| | | the combination of a C16:0 fatty acid unit and a |
| | | C22:6 fatty acid unit or the combination of a C18:2 fatty |
| | | acid unit and a C20:4 fatty acid unit. The mass-to- |
| | | charge ratio (m/z) of the ionised species is 806.6 Da |
| | | (+/−0.5 Da). |
| Phosphatidylcholine | 834.8 | Phosphatidylcholine (C18:0/C22:6) represents the sum |
| (C18:0/C22:6) | | parameter of glycerophosphorylcholines containing the |
| | | combination of a C18:0 fatty acid unit and a C22:6 fatty |
| | | acid unit. The mass-to-charge ratio (m/z) of the ionised |
| | | species is 834.6 Da (+/−0.5 Da). |
| Phosphatidylcholine | 768.8 | Phosphatidylcholine plasmalogenes represents the sum |
| plasmalogenes | | parameter of glycerophosphorylcholine plasmalogens. |
| | | The mass-to-charge ratio (m/z) of the ionised species is |
| | | 768.6 Da (+/−0.5 Da). |
| Pseudouridine | 217 | Pseudouridine which is present in human serum and if |
| | | detected with GC/MS analysis with application of an |
| | | electron impact mass spectrometry at 70 eV and after |
| | | derivatisation with 2% O-methylhydroxylamine- |
| | | hydrochlorid in pyridine and subsequently with N-methyl- |
| | | N-trimethylsilyltrifluoracetamid has the following characteristic |
| | | nominal masses (relative ratios): 217 (100 +/− 20%), |
| | | 73 (82 +/− 20%), 357 (21 +/− 20%), 147 (20 +/− 20%), |
| | | 218 (17 +/− 20%), 269 (8 +/− 20%), 424 (17 +/− 7%), 589 |
| | | (3 +/− 20%) |
| Sphingomyelin #1 | 723.6 | Sphingomyelin #1 represents the sum parameter of |
| | | sphingomyelins. The mass-to-charge ratio (m/z) of the |
| | | ionised species is 723.6 Da (+/−0.5 Da). |
| Sphingomyelin #2 | 815.8 | Sphingomyelin #2 represents the sum parameter of |
| | | sphingomyelins containing the combination of a d18:1 |
| | | long-chain base unit and a C24:0 fatty acid unit. The |
| | | mass-to-charge ratio (m/z) of the ionised species is |
| | | 815.8 Da (+/−0.5 Da). |
| TAG #2 | 695.6 | TAG #2 represents the sum parameter of triacylglycerols. |
| | | The mass-to-charge ratio (m/z) of the ionised species |
| | | is 695.6 Da (+/−0.5 Da). |
| TAG (C55H100O6) | 879.6 | TAG (C55H100O6) (e.g. C16:0, C18:1, C18:2) represents |
| (e.g. | | the sum parameter of triacylglycerols. The mass-to- |
| C16:0, C18:1, C18:2) | | charge ratio (m/z) of the ionised species is 879.6 Da |
| | | (+/−0.5 Da). |
| TAG (containing | 549.6 | TAG (containing C16:0/C16:1) represents the sum parameter |
| C16:0/C16:1) | | of triacylglycerols containing either the combination |
| | | of a C16:1 fatty acid unit and a C16:0 fatty acid unit |
| | | or the combination of a C18:1 fatty acid unit and a C14:0 |
| | | fatty acid unit. The mass-to-charge ratio (m/z) of the ionised |
| | | species is 549.6 Da (+/−0.5 Da). |
| TAG (containing | 599.6 | TAG (containing C18:2, C18:2) represents the sum parameter |
| C18:2, C18:2) | | of triacylglycerols containing the diacylglycero |
| | | subunit consisting of two C18:2 fatty acid units. The |
| | | mass-to-charge ratio (m/z) of the ionised species is |
| | | 599.6 Da (+/−0.5 Da). |
| Testosterone-17- | 367.4 | Testosterone-17-sulfate represents the sum parameter |
| sulfate | | of steroid sulfates. The mass-to-charge ratio (m/z) of the |
| | | ionised species is 367.4 Da (+/−0.5 Da). |

TABLE 15
Preferred Combinations of identified metabolites (Table 9) differing between
diabetic/obese and non-diabetic/obese at the pre-surgery time point t0 or post-surgery
time points t3 or t6 and the direction of difference indicated in parenthesis.

| Combibi- | Metabolite | | | | |
|---|---|---|---|---|---|
| nation | 1 + 2 | Metabolite 3 | Metabolite 4 | Metabolite 5 | Metabolite 6 |
| 1 | 1,5- | 5-Hydroxy-3- | Arginine | Asparagine | Citrulline (up) |
| | Anhydro- | indoleacetic | (down) | (up) | |
| | sorbitol | acid (up) | | | |
| | (down) + glucose | | | | |
| | (up) | | | | |
| 2 | 1,5- | Arginine | Asparagine | Citrulline (up) | Cysteine (up) |
| | Anhydro- | (down) | (up) | | |
| | sorbitol | | | | |
| | (down) + glucose | | | | |
| | (up) | | | | |
| 3 | 1,5- | Asparagine | Citrulline (up) | Cysteine (up) | erythro- |
| | Anhydro- | (up) | | | Sphingosine |
| | sorbitol | | | | (*1) (down) |
| | (down) + glucose | | | | |
| | (up) | | | | |
| 4 | 1,5- | Citrulline (up) | Cysteine (up) | erythro- | gamma- |
| | Anhydro- | | | Sphingosine | Linolenic acid |
| | sorbitol | | | (*1) (down) | (C18:cis[6,9,12]3) |
| | (down) + glucose | | | | (up) |
| | (up) | | | | |
| 5 | 1,5- | Cysteine (up) | erythro- | gamma- | Hypoxanthine |
| | Anhydro- | | Sphingosine | Linolenic acid | (up) |
| | sorbitol | | (*1) (down) | (C18:cis[6,9,12]3) | |
| | (down) + glucose | | | (up) | |
| | (up) | | | | |
| 6 | 1,5- | erythro- | gamma- | Hypoxanthine | Leucine (up) |
| | Anhydro- | Sphingosine | Linolenic acid | (up) | |
| | sorbitol | (*1) (down) | (C18:cis[6,9,12]3) | | |
| | (down) + glucose | | (up) | | |
| | (up) | | | | |
| 7 | 1,5- | gamma- | Hypoxanthine | Leucine (up) | Lysophosphatidylchloine |
| | Anhydro- | Linolenic acid | (up) | | (C18:2) |
| | sorbitol | (C18:cis[6,9,12]3) | | | (down) |
| | (down) + glucose | (up) | | | |
| | (up) | | | | |
| 8 | 1,5- | Hypoxanthine | Leucine (up) | Lysophosphatidylcholine | myo-Inositol- |
| | Anhydro- | (up) | | (C18:2) | 2-phosphate |
| | sorbitol | | | (down) | (up) |
| | (down) + glucose | | | | |
| | (up) | | | | |
| 9 | 1,5- | Leucine (up) | Lysophosphatidylcholine | myo-Inositol- | Nervonic acid |
| | Anhydro- | | (C18:2) | 2-phosphate | (C24:1) |
| | sorbitol | | (down) | (up) | (down) |
| | (down) + glucose | | | | |
| | (up) | | | | |
| 10 | 1,5- | Lysophosphatidylcholine | myo-Inositol- | Nervonic acid | Phenylalanine |
| | Anhydro- | (C18:2) | 2-phosphate | (C24:1) | (up) |
| | sorbitol | (down) | (up) | (down) | |
| | (down) + glucose | | | | |
| | (up) | | | | |
| 11 | 1,5- | myo-Inositol- | Nervonic acid | Phenylalanine | Proline (up) |
| | Anhydro- | 2-phosphate | (C24:1) | (up) | |
| | sorbitol | (up) | (down) | | |
| | (down) + glucose | | | | |
| | (up) | | | | |
| 12 | 1,5- | Nervonic acid | Phenylalanine | Proline (up) | Valine (up) |
| | Anhydro- | (C24:1) | (up) | | |
| | sorbitol | (down) | | | |
| | (down) + glucose | | | | |
| | (up) | | | | |
| 13 | 1,5- | Phenylalanine | Proline (up) | Valine (up) | Leucine (up) |
| | Anhydro- | (up) | | | |
| | sorbitol | | | | |
| | (down) + glucose | | | | |
| | (up) | | | | |
| 14 | 1,5- | Proline (up) | Valine (up) | Leucine (up) | |
| | Anhydro- | | | | |
| | sorbitol | | | | |
| | (down) + glucose | | | | |
| | (up) | | | | |
| 15 | 1,5- | Valine (up) | Leucine (up) | | |
| | Anhydro- | | | | |
| | sorbitol | | | | |
| | (down) + glucose | | | | |
| | (up) | | | | |
| 16 | 1,5- | Nervonic acid | Valine (up) | Leucine (up) | |
| | Anhydro- | (C24:1) | | | |
| | sorbitol | (down) | | | |
| | (down) + glucose | | | | |
| | (up) | | | | |
| 17 | 1,5- | Nervonic acid | Valine (up) | Proline (up) | |
| | Anhydro- | (C24:1) | | | |
| | sorbitol | (down) | | | |
| | (down) + glucose | | | | |
| | (up) | | | | |
| 18 | 1,5- | Nervonic acid | Leucine (up) | Valine (up) | Proline (up) |
| | Anhydro- | (C24:1) | | | |
| | sorbitol | (down) | | | |
| | (down) + glucose | | | | |
| | (up) | | | | |
| 19 | 1,5- | Nervonic acid | Lysophosphatidylcholine | | |
| | Anhydro- | (C24:1) | (C18:2) | | |
| | sorbitol | (down) | (down) | | |
| | (down) + glucose | | | | |
| | (up) | | | | |
| 20 | 1,5- | Nervonic acid | gamma- | | |
| | Anhydro- | (C24:1) | Linolenic acid | | |
| | sorbitol | (down) | (C18:cis[6,9,12]3) | | |
| | (down) + glucose | | (up) | | |
| | (up) | | | | |

All references referred to above are herewith incorporated by reference with respect to their entire disclosure content as well as their specific disclosure content explicitly referred to in the above description.

The invention will now be illustrated by the following Examples which are not intended to restrict or limit the scope of this invention.

### EXAMPLES

### Example 1

### Generation of Samples

The study included 14 prospectively recruited female subjects, aged 18-61 years and classified as severely obese (BMI>35 kg/m², median BMI=44.6, standard deviation of BMI=6.8) from the Department of Nutrition of the Hôtel-Dieu Hospital (Paris, France). 5 subjects were classified as diabetic/obese, and 9 as nondiabetic/obese. Preoperative evaluation included medical history, physical, nutritional, cardiopulmonary and psychological evaluations. All parameters were evaluated in the morning at the fasting state. Obese subjects were weight stable for at least 3 months before operation and met the criteria for obesity surgery, i.e. BMI≧40 kg/m² or ≧35 kg/m² with at least two significant co-morbidities (hypertension, type II diabetes or dyslipidemia). They were excluded from the protocol if there was evidence of acute or chronic inflammatory disease, infectious diseases, cancer and/or known alcohol consumption (>20 g per day), as well as other causes of liver diseases (viral hepatitis, hemochromatosis, Wilson's disease, auto-immune hepatitis, antitrypsine deficit). The Ethics Committees of the Hôtel-Dieu Hospital had approved the clinical investigations and all subjects had given informed consent.

Roux-en-Y gastric bypass (RGB) surgery, the most common and successful technique, was applied to all patients in this study. The surgery created a small stomach pouch to restrict food intake. A Y-shaped section of the small intestine was created by attaching the lower jejunum to the pouch to allow food to bypass the lower stomach, the duodenum and the first portion of the jejunum. Serum samples were collected for metabolite profiling and for standard clinical parameters. Metabolite profiling was performed for samples obtained before (0 months), 3 months after and 6 months after gastric bypass surgery. Standard clinical parameters were analyzed for the same samples and, in addition, for a time point 12 months after gastric bypass surgery.

### Example 2

### Metabolite Profiling

For mass spectrometry-based metabolite profiling analyses plasma samples were extracted and a polar and a non-polar fraction was obtained. For GC-MS analysis, the non-polar fraction was treated with methanol under acidic conditions to yield the fatty acid methyl esters. Both fractions were further derivatised with O-methyl-hydroxyamine hydrochloride and pyridine to convert Oxo-groups to O-methyloximes and subsequently with a silylating agent before analysis. In LC-MS analysis, both fractions were reconstituted in appropriate solvent mixtures. HPLC was performed by gradient elution on reversed phase separation columns. For mass spectrometric detection technology was applied as described in WO2003073464, which allows target and high sensitivity MRM (Multiple Reaction Monitoring) profiling in parallel to a full screen analysis. Steroids and their metabolites were measured by online SPE-LC-MS (Solid phase extraction-LC-MS). Catecholamins and their metabolites were measured by online SPE-LC-MS (Solid phase extraction-LC-MS) as for example described by Yamada et al. (Yamada H. Yamahara A. Yasuda S. Abe M. Oguri K. Fukushima S. Ikeda-Wada S. Dansyl chloride derivatization of methamphetamine: A method with advantages for screening and analysis of methamphetamine in urine. Journal of Analytical Toxicology. 26(1):17-22, 2002 Jan.-Feb.).

### Example 3

### Data Analysis

Following comprehensive analytical validation steps, the data for each analyte were normalized against data from pool samples. These samples were run in parallel through the whole process to account for process variability. To eliminate minor, potentially confounding effects and for statistical analysis, mixed linear models were used (based on log 10-transformed pool-normalized metabolite data). Factors were treatment (pre-surgery (reference), 3 and 6 months post-surgery), indication (diabetic/obese and nondiabetic/obese) and interaction between treatment and indication (optional, only included if positively contributing to model quality). Reference for indication was diabetic/obese in one model and nondiabetic/obese in a second model. To read out indication effects at the post-surgery time points, additional linear models were generated using either 3 months post-surgery or 6 months post-surgery as reference for factor treatment. From these linear models, ratios were derived indicating effect size and p-values of t-statistics indicating statistical significance. Regulation type was determined for each metabolite as “up” for increased (ratios >1) of the respective factor level vs. reference and “down” for decreased (ratios <1) of factor level vs. reference.

In order to generate tables 1A and 2 to 12 (see above), the following results from the linear models were used.
(1) Treatment (3 months vs. pre-surgery) in the nondiabetic/obese group
(2) Treatment (3 months vs. pre-surgery) in the diabetic/obese group (identical to (1) unless an interaction between treatment and indication was identified)
(3) Treatment (6 months vs. pre-surgery) in the nondiabetic/obese group
(4) Treatment (6 months vs. pre-surgery) in the diabetic/obese group (identical to (3) unless an interaction between treatment and indication was identified)
(5) Indication (diabetic/obese vs. nondiabetic/obese) at the pre-surgery time point or at 3 months post-surgery or at 6 months post-surgery

In another aspect of the analysis, a mixed linear model without interaction between treatment and indication was calculated. The model was based on log 10-transformed pool-normalized metabolite data. Factors were treatment (pre-surgery (reference), 3 and 6 months post-surgery) and indication (diabetic/obese and nondiabetic/obese). Reference for indication was nondiabetic/obese. From this linear model, ratios were derived indicating effect size and p-values of t-statistics indicating statistical significance. Regulation type was determined for each metabolite as “up” for increased (ratios >1) of the respective factor level vs. reference and “down” for decreased (ratios <1) of factor level vs. reference.

In order to generate table 1B, the following results from the linear models were used.

(1) Treatment (3 months vs. pre-surgery)

(2) Treatment (6 months vs. pre-surgery)

In addition, log 10-transformed metabolite data (pool-normalized ratios) were used for correlation analysis with selected clinical data (A-D,F: not log-transformed; E: log 10-transformed).
(A) Insulin sensitivity (QUICKI): 1/((log [fasting glucose])+(log [fasting insulin])).
(B) Body lean mass (in % of total body mass; estimated by DEXA (Dual-Energy X-ray Absorptiometry))
(C) Resting energy expenditure (REE, calculated according to: REE (kcal/d)=309+21.6×body lean mass (kg))
(D) Difference in insulin sensitivity (QUICKI) between 12 months post-surgery and pre-surgery time points
(E) Difference in body fat mass (in % of total body mass; estimated by DEXA) between 12 months post-surgery and pre-surgery time points
(F) Body fat mass (in % of total body mass; estimated by DEXA)

Depending on the question in focus, either metabolite data from all 3 time points (pre-surgery, 3 and 6 months after surgery) or only the pre-surgery time point (for predictive analysis) were used. From these linear regression analyses, R square (R²) values were calculated indicating explained variability and p-values of F-statistics indicating statistical significance.

1. A method of assessing whether gastric bypass therapy was successful in a subject comprising:
a) determining the amount of at least one biomarker selected from the group of biomarkers shown in any one of Tables 1A, 1B, 3 and 5 in a sample of said subject; and
b) comparing said amount to a reference, whereby it is to be diagnosed whether gastric bypass therapy was successful.

2. The method of claim 1, wherein said assessing further comprises predicting whether gastric bypass therapy was successful with respect to diabetes based on the comparison of at least one biomarker selected from the group of biomarkers as shown in Tables 2 and 3 to a reference.

3. The method of claim 1, wherein said assessing further comprises predicting whether gastric bypass therapy was successful with respect to obesity based on the comparison of at least one biomarker selected from the group of biomarkers as shown in Tables 4 and 5 to a reference.

4. The method of claim 1, wherein the gastric bypass therapy is “Roux-en-Y” bariatric surgery.

5. A method of predicting whether gastric bypass therapy will be beneficial for a subject in need thereof comprising:
a) determining the amount of at least one biomarker selected from the group of biomarkers shown in Tables 6 and 7 in a sample of said subject; and
b) comparing said amount to a reference, whereby it is to be predicted whether gastric bypass therapy will be beneficial.

6. The method of claim 5, wherein the gastric bypass therapy is “Roux-en-Y” bariatric surgery.

7. A method of diagnosing whether a supportive therapy accompanying gastric bypass therapy has beneficial effects on a subject in need thereof comprising:
a) determining the amount of at least one biomarker selected from the group of biomarkers shown in Table 8 in a sample of said subject; an
b) comparing said amount to a reference, it is to be determined whether the supplement diet has beneficial effects.

8. The method of claim 7, wherein said supportive therapy is selected from the group consisting of: nutritional therapy, a dietary supplement, a drug and combinations thereof.

9. The method of claim 7, wherein said gastric bypass therapy is “Roux-en-Y” bariatric surgery.

10. A method of diagnosing diabetes in a subject comprising:
a) determining the amount of at least one biomarker selected from the group of biomarkers shown in Tables 9 and 10 or a combination of biomarkers as recited in Table 15 in a sample of said subject; and
b) comparing said amount to a reference, whereby diabetes is to be diagnosed.

11. A method of diagnosing body lean mass in a subject comprising:
a) determining the amount of at least one biomarker selected from the group of biomarkers shown in Table 12 in a sample of said subject; and
b) comparing said amount to a reference, whereby the amount of body lean mass is to be diagnosed.

12. A method of diagnosing the energy state of a subject comprising
a) determining the amount of at least one biomarker selected from the group as shown in Table 11 in a sample of a subject; and
b) comparing said amount to a reference, whereby the energy state is to be identified.

13. A method for identifying a treatment against diabetes and/or obesity comprising:
a) determining the amount of at least one biomarker selected from the group of biomarkers shown in any one of Tables 1A, 1B, 3 and 5 in a sample of a subject to which the drug has been administered; and
b) comparing said amount to a reference, whereby the treatment is to be identified.

14. The method of claim 13, wherein a treatment against diabetes is to be identified by the comparison of at least one biomarker as shown in Tables 2 and 3 to a reference.

15. The method of claim 13, wherein a treatment against obesity is to be identified by the comparison of at least one biomarker as shown in Tables 4 and 5 to a reference.

16. The method of claim 13, wherein said treatment is selected from the group consisting of: administration of drugs, nutritional diets, dietary supplements, surgery, bariatric surgery, supporting physical activity, life-style recommendations and combinations thereof.
